(19) United States
(12) Patent Application Publication
(10) Pub. No.: US 2022/0390459 A1
(43) Pub. Date: Dec. 8, 2022

(54) RAPID, HIGH-INTENSITY CHEMILUMINESCENT DIOXETANES

(71) Applicant: Beckman Coulter, Inc., Brea, MI (US)

(72) Inventors: Khaledur Rashid, Troy, MI (US); Guoping Wang, Novi, MI (US); Barry A. Schoenfelner, Saugatuk, MI (US); Wenhua Xie, Novi, MI (US); Renuka de Silva, Northvulle, MI (US)

(21) Appl. No.: 17/772,269

(22) PCT Filed: Oct. 28, 2020
(86) PCT No.: PCT/US2020/057755

§ 371 Date: Apr. 27, 2022

Related U.S. Application Data
(60) Provisional application No. 62/926,985, filed on Oct. 28, 2019.

(51) Int. Cl. G01N 33/58; C07D 321/00; C07D 409/10; C07F 9/655; C07F 9/6558; C07H 15/26; C09K 11/07; G01N 21/76

(52) U.S. Cl. CPC G01N 33/582; C07D 321/00; C07D 409/10; C07F 9/65512; C07F 9/65586; C07H 15/26; C09K 11/07; G01N 21/76; C09K 2211/1018

(57) ABSTRACT

Described herein are 1,2-dioxetanes that are useful as chemiluminescent probes, diagnostic agents, and imaging agents. Also described herein are compositions containing such compounds and methods of using the same.

### CROSS-REFERENCE TO RELATED APPLICATIONS

This application claims the benefit of U.S. Provisional Appl. Ser. No. 62/926,985, filed Oct. 28, 2019, which is incorporated by reference as if fully set forth herein.

### BACKGROUND

Chemiluminescent dioxetanes are strained cyclic peroxides that can undergo rapid decomposition to generate an excited, transient species that subsequently decays to ground state via emission of light.

Such compounds are useful as luminescent probes in a range of assays, including enzyme activity assays, immunoassays, and DNA detection assays. Chemiluminescence-based assays can offer excellent sensitivity because unlike fluorescence and absorption-based assays, no light excitation is required.

Dioxetanes can be generated in situ at the time of their use or prepared in advance in stable form and then later activated. When generated in situ via oxidation of a precursor alkene, chemiluminescent dioxetanes can also function as a detection or imaging method for reactive oxygen species (ROS). An example of a stable chemiluminescent dioxetane is 4-methoxy-4-(3-phosphatephenyl)spiro[1,2-dioxetane-3,2′-adamantane]. This compound, also known as LUMIGEN® PPD, can be activated upon treatment with an alkaline phosphatase (ALP). ALP is an enzyme that catalyzes the hydrolysis of phosphate groups. Once activated, the resulting compound subsequently undergoes fragmentation of the 1,2-dioxatane ring and emits light, thus functioning as a luminescent probe in alkaline phosphatase-labeled assays.

Dioxetane compounds have been developed that are sensitive and strongly emissive under non-aqueous conditions. However, such compounds suffer from weak emissions in aqueous media and take a long time to reach maximum luminescence after contact with a desired analyte. Surfactant-based luminescence enhancers have been added to dioxetane probes to amplify weak emissions in aqueous environments but use of such enhancers is neither desirable nor suitable in various applications.

### SUMMARY

There is a need for dioxetanes that provide a rapid response to the presence of analytes. There is also a need for dioxetanes that are strongly emissive and suitable for use in aqueous environments, without the need for surfactant-based enhancers. Various compounds disclosed herein provide such features.

The present disclosure provides a compound of Formula I and salts thereof.

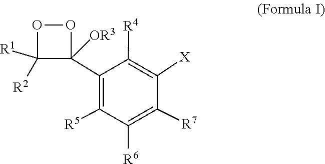

Each of R¹ and R² is independently C₃-C₁₀ alkyl, or R¹ and R² taken together with the carbon to which they are attached provide a C₅-C₁₀ cycloalkyl ring. R³ is C₁-C₁₀ alkyl, C₆-C₁₀ aryl, or heteroaryl.

Each of R⁴, R⁵, R⁶ and R⁷ is independently H, Q, X, hydroxy, halogen, amino, thio, C₁-C₁₀ alkyl, C₂-C₁₀ alkenyl, C₂-C₁₀ alkynyl, C₁-C₁₀ alkyloxy, C₁-C₁₀ alkylamino, C₁-C₁₀ trialkylammonium salt, C₁-C₁₀ alkylthio, C₂-C₁₀ acyl, C₁-C₁₀ alkyloxycarbonyl, C₁-C₁₀alkylaminocarbonyl, C₁-C₁₀alkylthiocarbonyl, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, C₁-C₁₀ alkylsulfinyl, C₁-C₁₀ alkylsulfonyl, arylthio, arylamino, arylsulfinyl, arylsulfonyl, arylcarbonyl, heteroarylcarbonyl, heteroaryloxy, heteroarylthio, heteroarylamino, heteroarylsulfinyl, heteroarylsulfonyl, cyano, nitro, trifluoromethyl, phosphonate, C₁-C₁₀ alkylphosphonate, C₁-C₁₀ alkylphosphinate, C₁-C₁₀ trialkylphosphonium salt, C₄-C₁₀ heterocycloamino, C₆-C₁₀ aryl, or π-excessive heteroaryl, and at least one of R⁴, R⁵, R⁶ and R⁷ is Q.

Q is a π-conjugated electron-donating group.

X is —OH, —O-G, an —O⁻ salt, or a boronate group.

G is an alcohol protecting group.

The present disclosure also provides an aqueous composition comprising one or more chemiluminescent dioxetane compound having a peak luminescent intensity of greater than 1000 photons/sec and a T_{1/2} of 3 minutes or less at 37° C. upon treatment with a pH 9.7 buffer, and wherein the composition is substantially free of surfactants-based luminescence enhancers.

The present disclosure also provides a method for determining the presence of an analyte in a sample, comprising contacting the sample with a compound of Formula I and monitoring the sample for luminescence.

Advantages, some of which are unexpected, are achieved by various embodiments of the present disclosure. Various compounds described herein can advantageously provide a rapid, high-intensity luminescent signal in non-aqueous media, aqueous media, or both. It is a significant advantage that assays involving such compounds can be performed faster than those with compounds lacking the features of the presently described compounds. Moreover, the compounds of the present disclosure provide increased intensity of luminescence, including in aqueous media. It is another advantage of the present compounds that aqueous compositions thereof can be free of surfactant-based luminescence enhancers. Due to such advantageous properties, various embodiments of the present disclosure can provide a method or kit that can detect an analyte in an aqueous or non-aqueous sample in under 3 minutes, under 1 minute, under 30 seconds, or in about 15 seconds or less.

### BRIEF DESCRIPTION OF THE DRAWINGS

FIG. 1 is a graph showing the time profile of chemiluminescence intensity of the compound of Example 1, using 20 μL of a 1 mg/mL sample of the compound in methanol which was triggered with 200 μL of amine-based buffer at 37° C.

FIG. 2 is a graph showing the time profile of chemiluminescence intensity of the compound of Example 2, using 10 μL of a 1 mg/mL sample of the compound in THF which was triggered with 200 μL of amine-based buffer at 37° C.

FIG. 3 is a graph showing the time profile of chemiluminescence intensity of the compound of Example 3, using 10 μL of a 1 mg/mL sample of the compound in THF which was triggered with 200 μL of amine-based buffer at 37° C.

FIG. 4 is a graph showing the time profile of chemiluminescence intensity of the compound of Example 4, using 10 μL of a 1 mg/mL sample of the compound in THF which was triggered with 200 μL of amine-based buffer at 37° C.

FIG. 5 is a graph showing the time profile of chemiluminescence intensity of the compound of Example 5, using 10 μL of a 0.1 mg/mL sample of the compound in dioxane which was triggered with 200 μL of amine-based buffer at 37° C.

FIG. 6 is a graph showing the time profile of chemiluminescence intensity of the compound of Example 7, using 10 μL of a 1 mg/mL sample of the compound in dioxane, further diluted with 90 μL water, which was triggered with 200 μL of amine-based buffer at 37° C.

FIG. 7 is a graph showing the time profile of chemiluminescence intensity of the compound of Example 8, using 10 μL of a 0.001 mg/mL sample of the compound in dioxane, further diluted with 90 μL water, which was triggered with 200 μL of amine-based buffer at 37° C.

FIG. 8 is a graph showing the time profile of chemiluminescence intensity of the compound of Example 9, using 10 μL of a 0.01 mg/mL sample of the compound in dioxane, further diluted with 90 μL water, which was triggered with 200 μL of amine-based buffer at 37° C.

FIG. 9 is a graph showing the time profile of chemiluminescence intensity of the compound of Example 10, using 10 μL of a 0.01 mg/mL sample of the compound in dioxane, further diluted with 90 μL water, which was triggered with 200 μL of amine-based buffer at 37° C.

FIG. 10 is a graph showing the time profile of chemiluminescence intensity of the compound of Example 11, using 10 μL of a 0.01 mg/mL sample of the compound in dioxane further diluted with 90 μL water, which was triggered with 200 μL of amine-based buffer at 37° C.

FIG. 11 is a graph showing the time profile of chemiluminescence intensity of the compound of Example 12, using 100 μL of a 1.25 mg/mL sample of the compound in 5 mg/mL TBE enhancer amine-based buffer with 10 μL of an alkaline phosphatase (AP8) at 37° C.

FIG. 12 is a graph showing the time profile of chemiluminescence intensity of the compound of Example 13, using 100 μL of a 0.125 mg/mL sample of the compound in 2.5 mg/mL TBE enhancer amine-based buffer with 10 μL of an alkaline phosphatase (AP8) at 37° C.

FIG. 13 is a graph showing the time profile of chemiluminescence intensity of the compound of Example 14, using 100 μL of a 0.25 mg/mL sample of the compound in 5 mg/mL TBE enhancer amine-based buffer with 10 μL of an alkaline phosphatase (AP8) at 37° C.

FIG. 14 is a graph showing the time profile of chemiluminescence intensity of the compound of Example 15, using 100 μL of a 0.25 mg/mL sample of the compound in 5 mg/mL TBE enhancer amine-based buffer with 10 μL of an alkaline phosphatase (AP8) at 37° C.

FIG. 15 is a graph showing the time profile of chemiluminescence intensity of the compound of Example 16, using 100 μL of a 0.25 mg/mL sample of the compound in 2.5 mg/mL TBE enhancer amine-based buffer with 10 μL of an alkaline phosphatase (AP8) at 37° C.

FIG. 16 is a graph showing the time profile of chemiluminescence intensity of the compound of Example 16, using 100 μL of a 0.1 mg/mL sample of the compound in water with 10 μL of an alkaline phosphatase (AP4) and 300 μL of 5 mg/mL TBE enhancer amine-based buffer at 37° C.

FIG. 17 is a graph showing the time profile of chemiluminescence intensity of the compound of Example 17, using 100 μL of a 0.2 mg/mL of the compound in amine-based buffer without poly(vinylbenzyl tributylphosphonium chloride) (TBE) and 10 μL of an alkaline phosphatase (AP9) at 37° C.

FIG. 18 is a graph showing the time profile of chemiluminescence intensity of the compound of Example 12 (Lumigen® PPD), using 100 μL of a 2 mg/mL of the compound in amine-based buffer without TBE enhancer and with 20 μL of an alkaline phosphatase (AP8) at 37° C.

### DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

The compounds of the present disclosure are useful in chemiluminescent applications, such as assays and chemical probes.

The present disclosure provides a compound of Formula I, or a salt thereof.

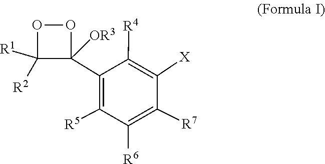

Each of R¹ and R² is independently C₃-C₁₀ alkyl, or R¹ and R² taken together with the carbon to which they are attached provide a C₅-C₁₀ cycloalkyl ring, e.g., a monocyclic, bicyclic, or tricyclic ring. R¹ and R² can be substituted or unsubstituted. In various embodiments, R¹ and R² are linked such that they provide, together with the carbon to which they are attached, a spirocyclic bridged bicyclo or tricyclo group. For example, R¹ and R² taken together with the carbon to which they are attached can be a spirocyclic adamantane, norbornane, or bornane.

R³ is C₁-C₁₀ alkyl, C₆-C₁₀ aryl, or heteroaryl, each of which is optionally substituted. R³ can be substituted or unsubstituted. For example, R³ can be unsubstituted C₁-C₁₀ alkyl or C₁-C₁₀ alkyl substituted with one or more halogen, hydroxy, amino, thio, alkoxy, alkylamino, alkylthio, sulfate, or carboxylate.

Each of R⁴, R⁵, R⁶ and R⁷ is independently H, Q, X, hydroxy, halogen, amino, thio, C₁-C₁₀ alkyl, C₂-C₁₀ alkenyl, C₂-C₁₀ alkynyl, C₁-C₁₀ alkyloxy, C₁-C₁₀ alkylamino, C₁-C₁₀ trialkylammonium salt, C₁-C₁₀ alkylthio, C₂-C₁₀ acyl, C₁-C₁₀ alkyloxycarbonyl, C₁-C₁₀alkylaminocarbonyl, C₁-C₁₀alkylthiocarbonyl, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, C₁-C₁₀ alkylsulfinyl, C₁-C₁₀ alkylsulfonyl, arylthio, arylamino, arylsulfinyl, arylsulfonyl, arylcarbonyl, heteroarylcarbonyl, heteroaryloxy, heteroarylthio, heteroarylamino, heteroarylsulfinyl, heteroarylsulfonyl, cyano, nitro, trifluoromethyl, phosphonate, C₁-C₁₀ alkylphosphonate, C₁-C₁₀ alkylphosphinate, C₁-C₁₀ trialkylphosphonium salt, C₄-C₁₀ heterocycloamino, C₆-C₁₀ aryl, or π-excessive heteroaryl, and at least one of R⁴, R⁵, R⁶ and R⁷ is Q. Each of R⁴, R⁵, R⁶ and R⁷ can be substituted or unsubstituted.

In various embodiments, R⁴, R⁵, R⁶ and R⁷ are each independently selected from the group H, Q, X, halogen, C₁-C₁₀ alkyl, hydroxy, C₁-C₁₀ alkyloxy, amino, C₁-C₁₀ alkylamino, thio, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, and heteroarylamino, wherein at least one of R⁴, R⁵, R⁶ and R⁷ is Q.

In various further embodiments, R⁴, R⁵, R⁶ and R⁷ provide a net electron donating effect on the aromatic ring to which they are attached. For example, the aromatic ring to which X, Q, R⁴, R⁵, R⁶ and R⁷ attach is electron-enriched relative to an otherwise identical compound in which Q, R⁴, R⁵, R⁶ and R⁷ is H.

In some embodiments, exactly one, two or three of R⁴, R⁵, R⁶ and R⁷ are X; exactly one, two or three of R⁴, R⁵, R⁶ and R⁷ is Q; or any combination thereof. In some further such embodiments, the remainder of R⁴, R⁵, R⁶ and R⁷ that is not X or Q is H. For example each of R⁴, R⁵, and R⁶ can be H while R⁷ is Q.

Q is a π-conjugated group, an electron donating group, or both. In various embodiments, Q is a π-conjugated electron donating group. In various embodiments, Q is C₂-C₁₀ alkenyl, C₂-C₁₀ heterocycloalkenyl, C₆-C₁₀ aryl, or heteroaryl. Q can be substituted or unsubstituted. In some embodiments, when Q is C₂-C₁₀ alkenyl it is substituted with one or more electron-donating groups, it is free of electron-withdrawing groups, or both. In various embodiments, when Q is C₂-C₁₀ alkenyl the vinylic positions and allylic positions, if present, are unsubstituted In some further embodiments. For example, Q can be unsubstituted vinyl. In some embodiments, when Q is C₆-C₁₀ aryl it is substituted with one or more electron-donating groups, it is free of electron-withdrawing groups, or both. For example, Q can be unsubstituted phenyl, phenyl substituted with one or more electron-donating groups, or phenyl substituted with one or more substituents selected from a group consisting of electron-donating substituents. As another example, Q can be vinyl or phenyl substituted with substituents such that the net effect of the substituents is an electron-donating effect. In a further example, Q is a π-excessive heteroaryl such as thiophenyl, furanyl, pyrrolyl, benzothiophenyl, benzofuranyl, or indolyl. In certain embodiments, Q is substituted or unsubstituted thiophen-2-yl or thiophen-3-yl.

X is —OH, —O-G, an —O⁻ salt, or a boronate group. In various embodiments, X is a group that generates an oxy anion upon chemical or enzymatic trigger. When X is a boronate group, it has the structure:

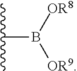

R⁸ and R⁹ of the boronate group is each independently H or C₁-C₁₀ alkyl, or R⁸ and R⁹ taken together with the boronate to which they are attached is a C₂-C₁₀ cyclic boronate ester. For example, X can 4,4,5,5-tetramethyl-1,3,2-dioxaborolanyl or —B(OH)₂.

In various embodiments, X is —O-G, wherein G is an alcohol protecting group, an analyte-responsive group, or both. For example, G can be trialkylsilyl, alkylarylsilyl, arylsulfonyl, dioxobenzyl, trityl, alkylcarbonate, phosphoryl, dihydropyranyl, tetrahydropyranyl, dihydrofuranyl, tetrahydrofuranyl, pyranosyl, pyranuronyl, furanosyl, acyl, benzoyl, or benzyl. In some embodiments, G is pyranosyl or pyranuronyl such as galactosyl, glucosyl, or glucuronyl. In further such embodiments, G is β-galactosyl, β-glucosyl, or β-glucuronyl. G can also be a phosphorous-containing group, such as a phosphate, phosphonate, and the like. For example, G can be —PO₃H₂ or a salt or ester thereof. In further embodiments, G is 2,4-dinitrobenzenesulfonyl, 3,4,6-trimehyl-2,5-dioxobenzyl, 4-azidobenzyloxy, tert-butyldimethylsilyl, acetyl, pivaloyl, an enzyme-cleavable moiety. For example, G can be a phosphatase-cleavable moiety or a peptidase-cleavable moiety. G can also comprise a divalent fragmentable linker having a pendant protecting group such that removal of the pendant protecting group triggers fragmentation of the linker and removal of the protecting group, G. Thus, G can contain a divalent fragmentable linker such as 4-aminobenzyl, 4-(alkylamino)benzyl, 4-oxybenzyl, 4-(oxymethyl)benzyl, oxymethyl, aminomethyl, alkylaminomethyl, and the like, together with a terminal protecting group such as trialkylsilyl, alkylarylsilyl, arylbenzenesulfonyl, dioxobenzyl, trityl, alkylcarbonate, phosphoryl, dihydropyranyl, tetrahydropyranyl, dihydrofuranyl, tetrahydrofuranyl, pyranosyl, pyranuronyl, furanosyl, acyl, benzoyl, benzyl or boronate group.

Examples of X and —O-G include the following structures:

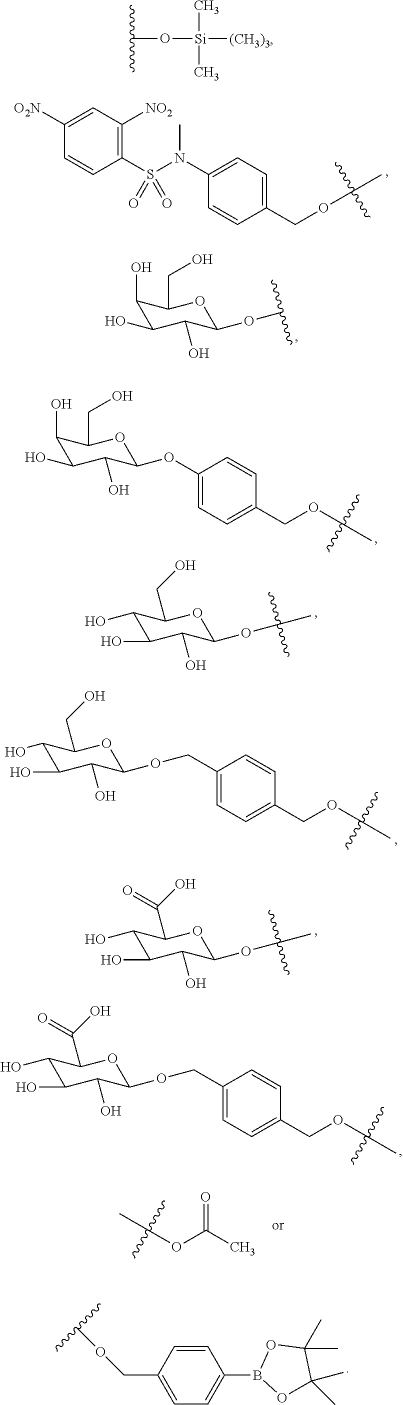

The present disclosure also provides a compound of Formula II, or a salt thereof.

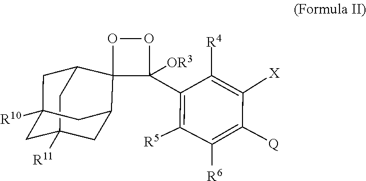

Each of R¹⁰ and R¹¹ is independently H, halogen, C₁-C₁₀ alkyl, C₂-C₁₀ alkenyl, C₆-C₁₀ aryl. In some embodiments, R¹⁰ and R¹¹ is independently H or halogen.

The present disclosure also provides a compound of Formula Ila and IIb, or a salt thereof.

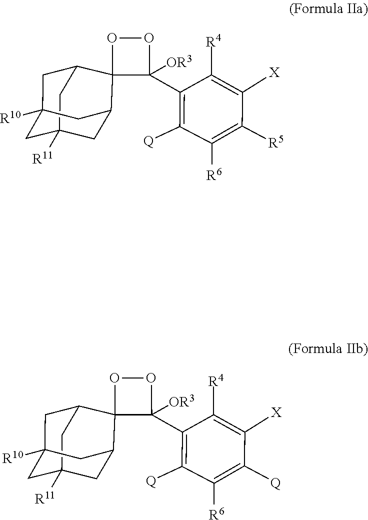

Each of R¹⁰ and R¹¹ is independently H, halogen, C₁-C₁₀ alkyl, C₂-C₁₀ alkenyl, C₆-C₁₀ aryl. In some embodiments, R¹⁰ and R¹¹ is independently H or halogen.

The present disclosure further provides a compound of Formula III, or a salt thereof.

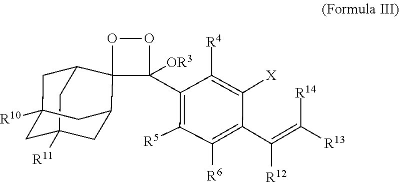

Each of R¹² and R¹³ is independently H, halogen, C₁-C₁₀ alkyl, C₂-C₁₀ alkenyl, C₂-C₁₀ alkynyl, C₁-C₁₀ alkyloxy, C₁-C₁₀ alkylamino, C₁-C₁₀ trialkylammonium salt, C₁-C₁₀ alkylthio, C₂-C₁₀ acyl, C₁-C₁₀ alkyloxycarbonyl, C₁-C₁₀ alkylaminocarbonyl, C₁-C₁₀ alkylthiocarbonyl, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, C₁-C₁₀ alkylsulfinyl, C₁-C₁₀ alkylsulfonyl, arylthio, arylamino, arylsulfinyl, arylsulfonyl, arylcarbonyl, heteroarylcarbonyl, heteroaryloxy, heteroarylthio, heteroarylamino, heteroarylsulfinyl, heteroarylsulfonyl, cyano, nitro, trifluoromethyl, phosphonate, C₁-C₁₀ alkylphosphonate, C₁-C₁₀ alkylphosphinate, C₁-C₁₀ trialkylphosphonium salt, C₄-C₁₀ heterocycloamino, C₆-C₁₀ aryl, or π-excessive heteroaryl, or R¹² and R¹³ taken together with the carbons to which they are attached provide a C₅-C₁₀ cycloalkenyl, C₂-C₁₀ heterocycloalkenyl, C₆-C₁₀ aryl, or π-excessive heteroaryl. In some embodiments, R¹² and R¹³ is independently H, C₁-C₁₀ alkyl, C₂-C₁₀ alkenyl, C₆-C₁₀ aryl, or π-excessive heteroaryl, or R¹² and R¹³ taken together with the carbons to which they are attached provide a C₅-C₁₀ cycloalkenyl, C₂-C₁₀ heterocycloalkenyl, C₆-C₁₀ aryl, or π-excessive heteroaryl, or R¹² and R¹³ taken together with the carbons to which they are attached provide a C₅-C₁₀ cycloalkenyl, C₂-C₁₀ heterocycloalkenyl, C₆-C₁₀ aryl, or π-excessive heteroaryl. In some further embodiments, R¹² and R¹³ is independently H, C₁-C₁₀ alkyl, C₂-C₁₀ alkenyl, C₆-C₁₀ aryl, or π-excessive heteroaryl.

R¹⁴ is H, halogen, C₁-C₁₀ alkyl, C₂-C₁₀ alkenyl, C₁-C₁₀ alkyloxy, C₁-C₁₀ alkylamino, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, or heteroarylamino, C₄-C₁₀ heterocycloamino, C₆-C₁₀ aryl, or π-excessive heteroaryl. In some embodiments, R¹⁴ is H, C₁-C₁₀ alkyl, C₂-C₁₀ alkenyl, C₆-C₁₀ aryl, or π-excessive heteroaryl.

R¹², R¹³ and R¹⁴ together have a net electron donating effect on the phenyl ring to which X is attached. For example, the aromatic ring to which X is attached is electron-enriched relative to an otherwise identical compound in which R¹², R¹³ and R¹⁴ are H.

In various embodiments, at least one or two of R¹², R¹³ and R¹⁴ is H.

The present disclosure further provides a compound of Formula IIIa and IIIb, or a salt thereof.

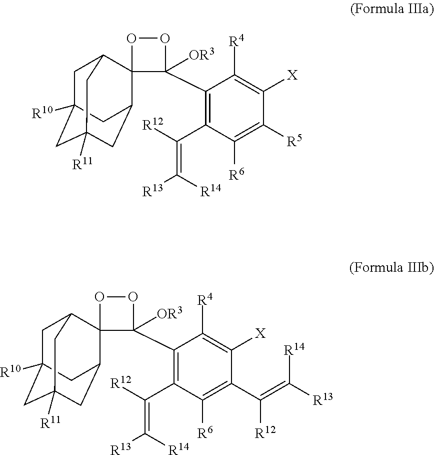

Each of R¹² and R¹³ is independently H, halogen, C₁-C₁₀ alkyl, C₂-C₁₀ alkenyl, C₂-C₁₀ alkynyl, C₁-C₁₀ alkyloxy, C₁-C₁₀ alkylamino, C₁-C₁₀ trialkylammonium salt, C₁-C₁₀ alkylthio, C₂-C₁₀ acyl, C₁-C₁₀ alkyloxycarbonyl, C₁-C₁₀ alkylaminocarbonyl, C₁-C₁₀ alkylthiocarbonyl, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, C₁-C₁₀ alkylsulfinyl, C₁-C₁₀ alkylsulfonyl, arylthio, arylamino, arylsulfinyl, arylsulfonyl, arylcarbonyl, heteroarylcarbonyl, heteroaryloxy, heteroarylthio, heteroarylamino, heteroarylsulfinyl, heteroarylsulfonyl, cyano, nitro, trifluoromethyl, phosphonate, C₁-C₁₀ alkylphosphonate, C₁-C₁₀ alkylphosphinate, C₁-C₁₀ trialkylphosphonium salt, C₄-C₁₀ heterocycloamino, C₆-C₁₀ aryl, or π-excessive heteroaryl, or R¹² and R¹³ taken together with the carbons to which they are attached provide a C₅-C₁₀ cycloalkenyl, C₂-C₁₀ heterocycloalkenyl, C₆-C₁₀ aryl, or π-excessive heteroaryl. In some embodiments, R¹² and R¹³ is independently H, C₁-C₁₀ alkyl, C₂-C₁₀ alkenyl, C₆-C₁₀ aryl, or π-excessive heteroaryl, or R¹² and R¹³ taken together with the carbons to which they are attached provide a C₅-C₁₀ cycloalkenyl, C₂-C₁₀ heterocycloalkenyl, C₆-C₁₀ aryl, or π-excessive heteroaryl, or R¹² and R¹³ taken together with the carbons to which they are attached provide a C₅-C₁₀ cycloalkenyl, C₂-C₁₀ heterocycloalkenyl, C₆-C₁₀ aryl, or π-excessive heteroaryl. In some further embodiments, R¹² and R¹³ is independently H, C₁-C₁₀ alkyl, C₂-C₁₀ alkenyl, C₆-C₁₀ aryl, or π-excessive heteroaryl.

R¹⁴ is H, halogen, C₁-C₁₀ alkyl, C₂-C₁₀ alkenyl, C₁-C₁₀ alkyloxy, C₁-C₁₀ alkylamino, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, or heteroarylamino, C₄-C₁₀ heterocycloamino, C₆-C₁₀ aryl, or π-excessive heteroaryl. In some embodiments, R¹⁴ is H, C₁-C₁₀ alkyl, C₂-C₁₀ alkenyl, C₆-C₁₀ aryl, or π-excessive heteroaryl.

R¹², R¹³ and R¹⁴ together have a net electron donating effect on the phenyl ring to which X is attached. For example, the aromatic ring to which X is attached is electron-enriched relative to an otherwise identical compound in which R¹², R¹³ and R¹⁴ are H.

In various embodiments, at least one or two of R¹², R¹³ and R¹⁴ is H.

The present disclosure further provides a compound according to one or more of the following formulae:

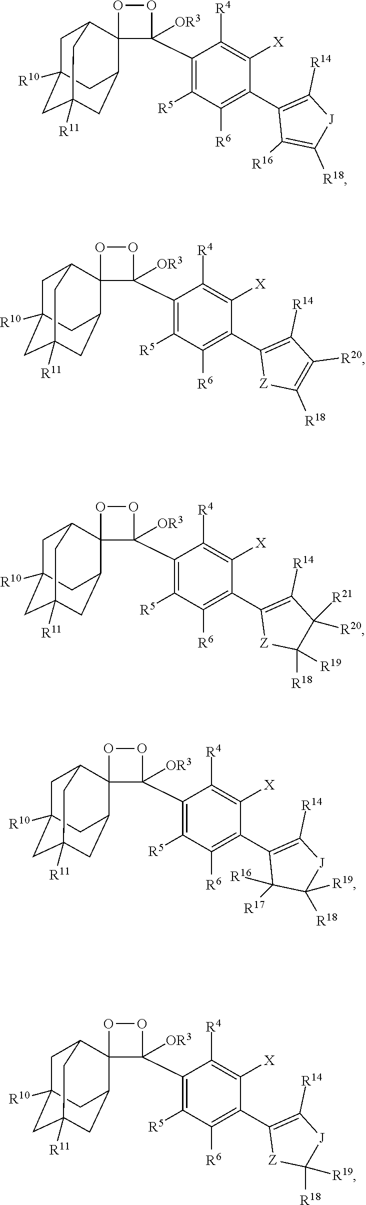

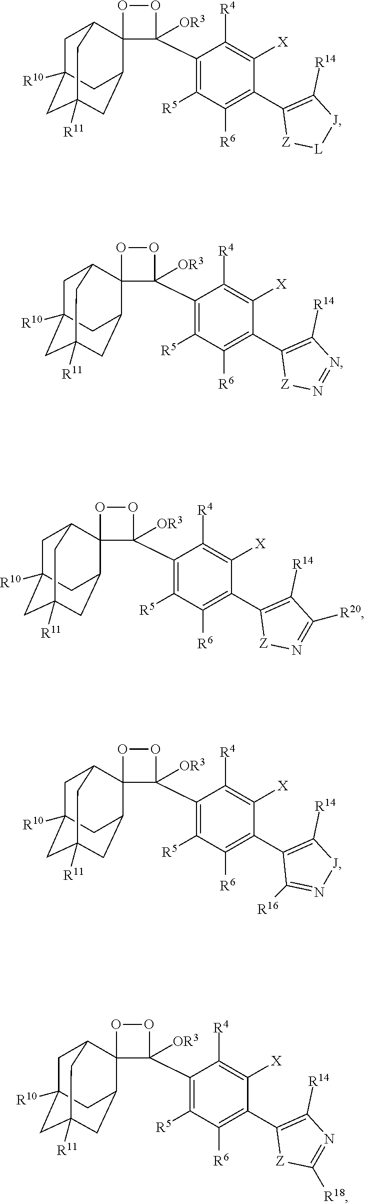

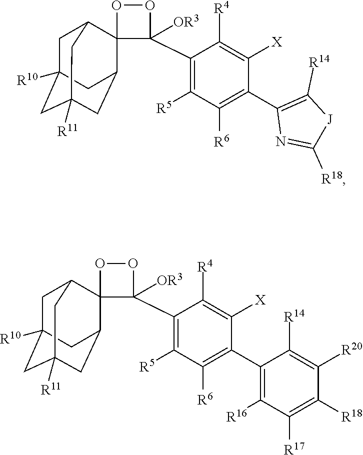

or a salt thereof.

The present disclosure further provides a compound according to one or more of the following formulae:

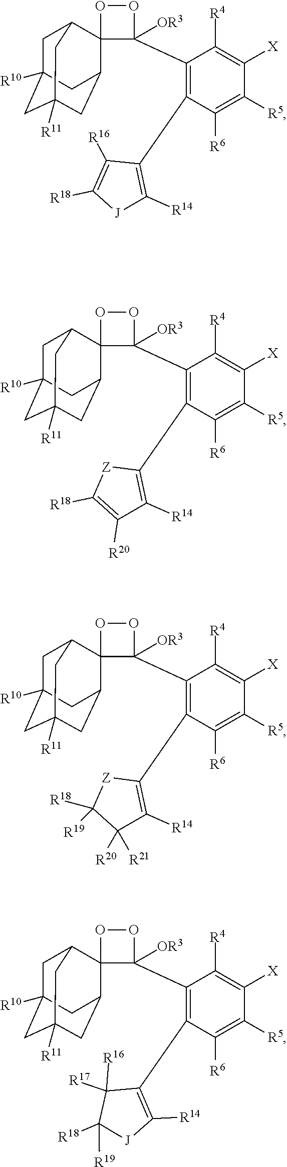

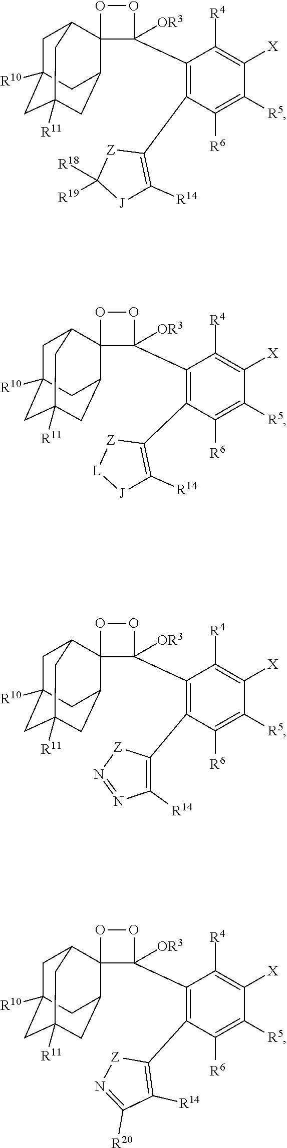

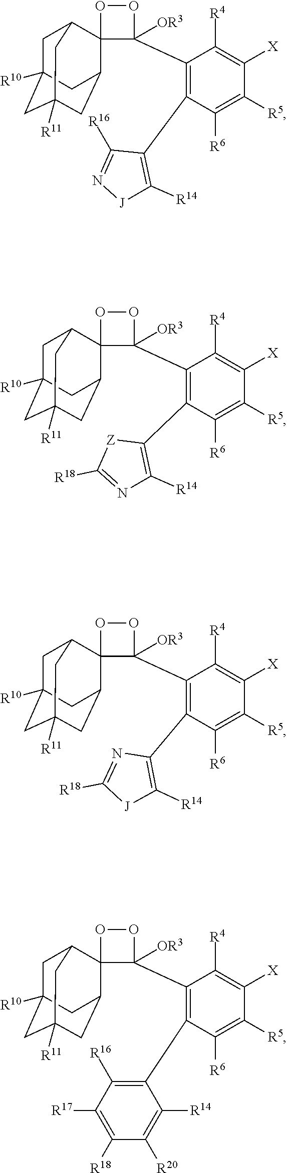

or a salt thereof.

Each of Z, L and J is S, O, Se, NR¹⁵, or (CR¹⁶R¹⁷)ₙ, wherein each R¹⁵ is independently H, alkyl, acyl, benzyl, alkyloxycarbonyl, arylsulfonyl; and each of R¹⁴, R¹⁶, R¹⁷, R¹⁸, R¹⁹, R²⁰ and R²¹, if present, is independently H, halogen, C₁-C₁₀ alkyl, C₂-C₁₀ alkenyl, C₁-C₁₀ alkyloxy, C₁-C₁₀ alkylamino, C₁-C₁₀ trialkylammonium salt, C₁-C₁₀ alkylthio, C₂-C₁₀ acyl, C₁-C₁₀ alkyloxycarbonyl, C₁-C₁₀ alkylaminocarbonyl, C₁-C₁₀ alkylthiocarbonyl, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, C₁-C₁₀ alkylsulfinyl, C₁-C₁₀ alkylsulfonyl, arylthio, arylamino, arylsulfinyl, arylsulfonyl, arylcarbonyl, heteroarylcarbonyl, heteroaryloxy, heteroarylthio, heteroarylamino, heteroarylsulfinyl, heteroarylsulfonyl, cyano, nitro, trifluoromethyl, phosphonate, C₁-C₁₀ alkylphosphonate, C₁-C₁₀ alkylphosphinate, C₁-C₁₀ trialkylphosphonium salt, C₄-C₁₀ heterocycloamino, C₆-C₁₀ aryl, or π-excessive heteroaryl, or any two of R¹⁴, R¹⁶, R¹⁷, R¹⁸ and R²⁰ taken together with the carbons to which they are attached provide a C₅-C₁₀ cycloalkenyl, C₂-C₁₀ heterocycloalkenyl, C₆-C₁₀ aryl, or π-excessive heteroaryl.

In some embodiments, each of R¹⁴, R¹⁶, R¹⁷, R¹⁸, R¹⁹, R²⁰ and R²¹, if present, is independently H, halogen, C₁-C₁₀ alkyl, C₂-C₁₀ alkenyl, C₁-C₁₀ alkyloxy, C₁-C₁₀ alkylamino, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, or heteroarylamino, C₄-C₁₀ heterocycloamino, C₆-C₁₀ aryl, or π-excessive heteroaryl, or any two of R¹, R¹⁶, R¹⁷, R¹⁸ and R²⁰ taken together with the carbons to which they are attached provide a C₅-C₁₀ cycloalkenyl, C₂-C₁₀ heterocycloalkenyl, C₆-C₁₀ aryl, or π-excessive heteroaryl.

In various embodiments at least one of R¹⁴, R¹⁶, R¹⁷, R¹⁸ and R²⁰ is an electron donating group. In other embodiments, each of R¹⁴, R¹⁶, R¹⁸ and R²⁰, if present, is hydrogen.

In any of the foregoing compounds, including the compounds of Formula I, II, IIa, III, IIIa, IIIb, and XII (below) R⁴ or R⁵ can be C₁-C₁₀ alkyl (e.g., CH₃) or halo (e.g., chloro). Further, R¹² in any of the foregoing compounds, including compounds of the Formula XII-XIV can be C₁-C₁₀ alkyl (e.g., CH₃).

The present disclosure further provides a compound according to Formula (IV)-(XV), or a salt thereof.

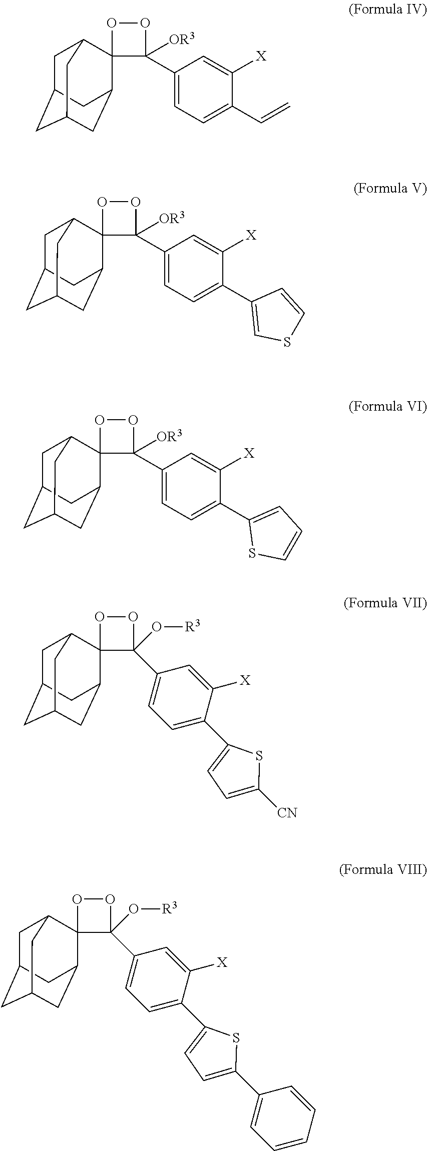

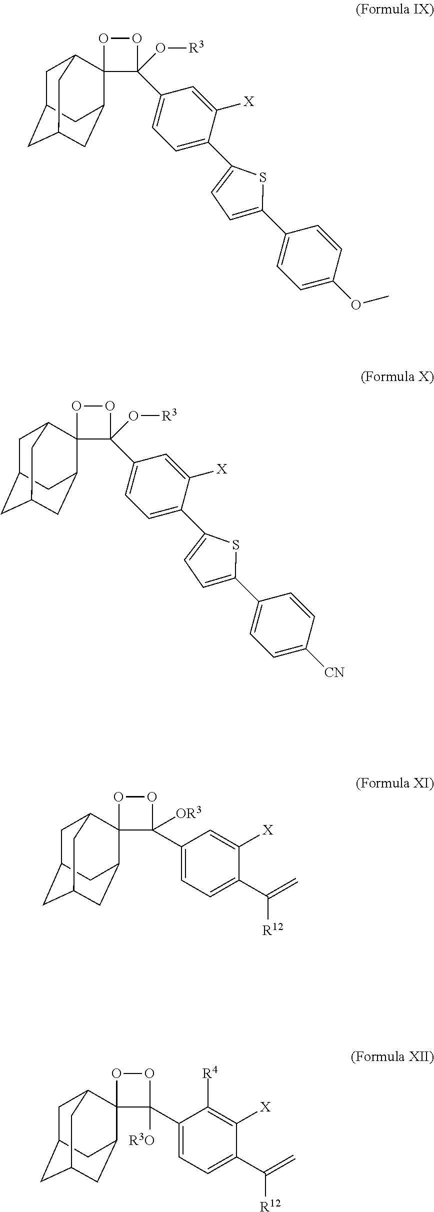

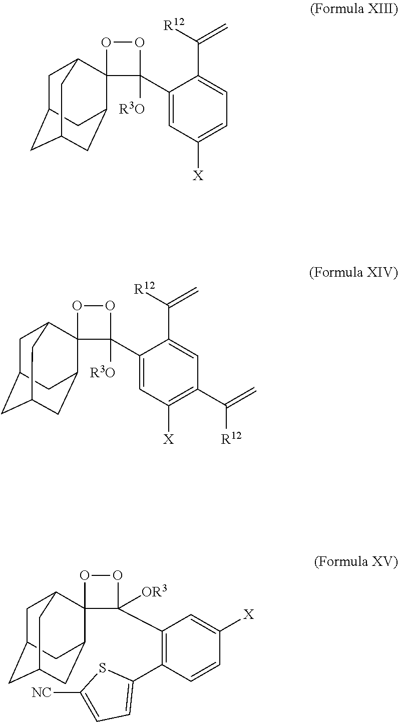

The present disclosure provides a compound according to one or more of the following structures:

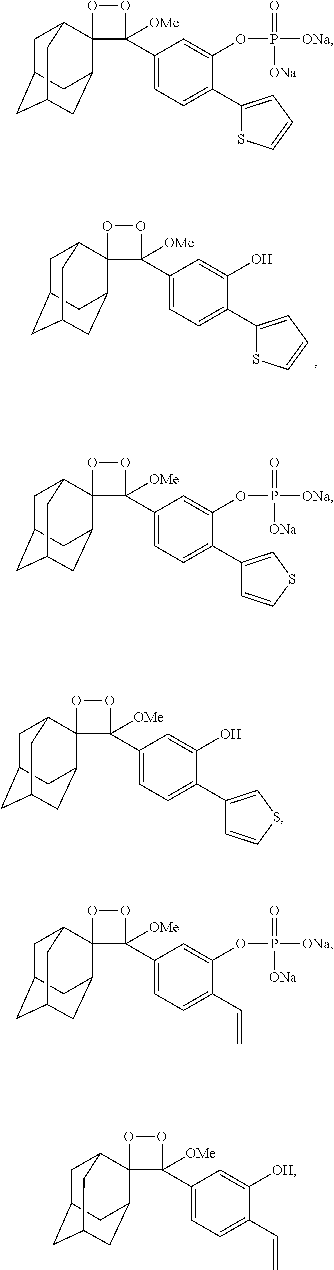

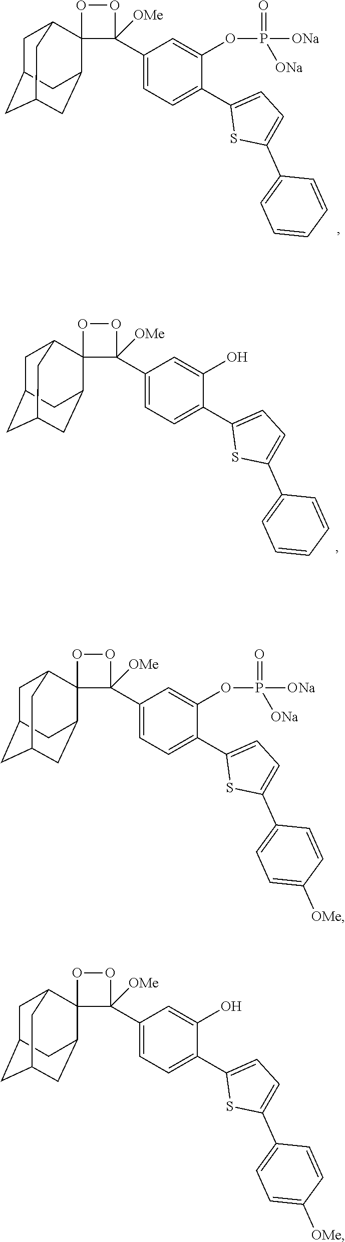

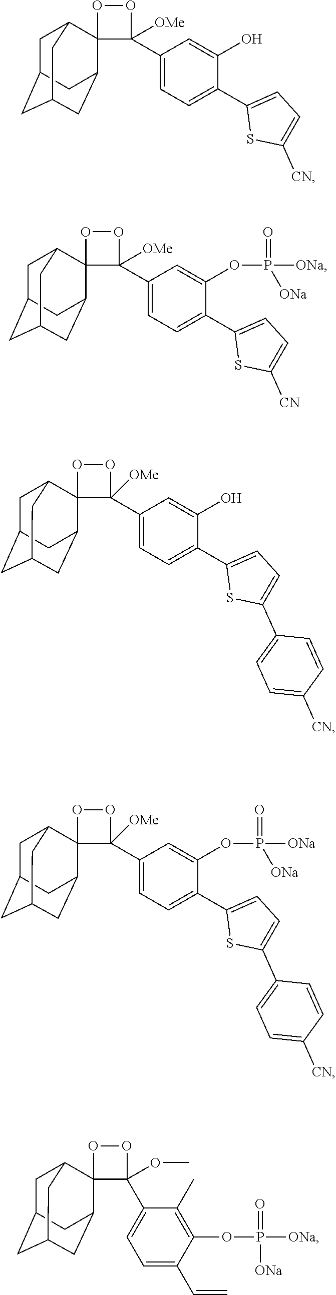

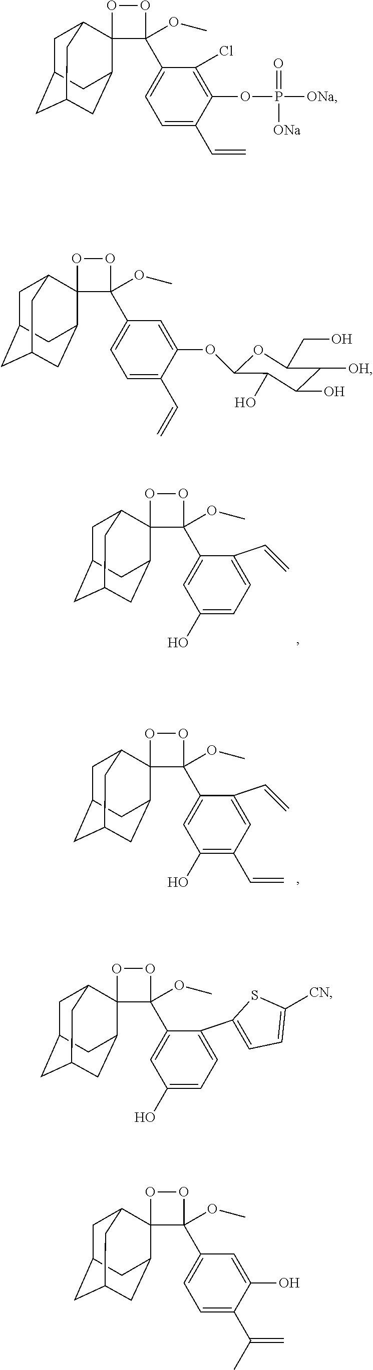

or a salt thereof.

The present disclosure provides a composition comprising one or more of the compounds described herein, an olefin precursor thereof, or salt thereof. An olefin precursor provides any compound described herein (e.g., a compound of Formula I, II, IIa, III, IIIa, IlIb, and IV-XV) upon treatment with an analyte, an oxidizing agent, an alkaline phosphatase, or photooxidation conditions.

The composition can be an aqueous composition or a non-aqueous composition. The composition can be mixture of both aqueous and non-aqueous solvents. In various embodiments, the composition is substantially free of surfactant-based luminescence enhancers, surfactants, or both. For example, the composition can be substantially free of surfactants having a tail group that is either an acyclic alkyl group (e.g., an acyclic group of at least eight carbons) or an aromatic group (e.g., an aromatic group comprising at least six carbons) and a head group that is one or more quaternary ammonium salt, pyridinium salt, quaternary phosphonium surfactant salt, ethyleneglycol, or fluorescein. As a further example, the composition can be substantially free of cetrimonium bromide (CTAB), cetylpyridinium chloride (CPC), benzalkonium chloride (BAC), benzethonium chloride (BZT), dimethyldioctadecylammonium chloride, dioctadecyldimethylammonium bromide (DODAB), α′-tributylphosphonium-p-xylene dichloride, poly(vinylbenzyl tributylphosphonium chloride) (TBE), poly(vinylbenzyl trioctylphosphonium chloride), Triton X-100, Tween surfactants, surfactants having a long alkyl chain having a polyethyleneglycol head, Brij® surfactants, IGEPAL® surfactants, octylphenoxypolyethoxyethanol, and the like. The composition can be free of fluorescein-containing compounds such as N-(3′,6′-dihydroxy-3-oxo-3H-spiro[isobenzofuran-1,9′-xanthen]-5-yl)tetradecanamide and other fluorescein-containing surfactants.

In various embodiments, the composition contains a buffer solution. The buffer solution can, but need not necessarily be, an alkaline or amine-based buffer solution. An example amine-based buffer is 221 buffer available from Sigma-Aldrich (St. Louis, Mo.). Likewise, the composition can, but need not necessarily have a basic pH. For example, composition can have a pH of about 4 to 12, about 5 to 12, about 6 to 12, about 7 to 12, about 8 to 12, about 9 to 12, about 10 to 12, about 4 to 11, about 4 to 10, about 4 to 9, about 4 to about 8, about 4 to 7, about 4 to 6, or about 4 to 5. The pH of the composition can be selected based whether luminescence is intended to trigger immediately upon analyte-triggered removal as is typically the case for alkaline pH values or the pH of the composition can be acidic so as to luminesce upon treatment with base.

In various embodiments, the composition has a peak luminescent intensity of greater than 1,000 photons/sec and a T_{1/2} of 3 minutes or less at 37° C. upon treatment with pH 9.7 buffer. In various examples, the peak luminescent intensity can be greater than about 2,000 photons/sec, about 3,000 photons/sec, about 4,000 photons/sec, about 5,000 photons/sec, about 6,000 photons/sec, about 7,000 photons/sec, about 8,000 photons/sec, about 9,000 photons/sec, or greater than about 10,000 photons/sec, and the T_{1/2} can be about 3 minutes or less, about 2 minutes or less, about 1 minute or less, about 30 seconds or less, about 20 seconds or less, about 15 seconds or less, or about 10 seconds or less. For example, the present disclosure provides an aqueous composition comprising one or more dioxetane compounds and having a peak luminescent intensity of greater than 1000 photons/sec and a T_{1/2} of 3 minutes or less at 37° C. upon treatment with pH 9.7 buffer, wherein the composition is substantially free of surfactant-based luminescence enhancers.

The present disclosure also provides a method of detecting an analyte in a sample, comprising contacting the sample with one or more of the compounds described herein, an olefin precursor thereof, a salt thereof, or a composition comprising the same, and then monitoring the sample for luminescence. In various embodiments, the method involves measuring the intensity of a resulting luminescence and correlating the intensity to the presence of the analyte.

In some embodiments, the method further involves increasing the pH of the sample. For example, the pH can be adjusted to 7 or higher, 8 or higher, 9 or higher, 10 or higher or 11 or higher.

In various embodiments, the analyte is detected in about 3 minutes or less, about 2 minutes or less, about 1 minute or less, about 30 seconds or less, about 20 seconds or less, about 15 seconds or less, or about 10 seconds or less. For example, the sample can be monitored for about 3 minutes or less, about 2 minutes or less, about 1 minute or less, about 30 seconds or less, about 20 seconds or less, about 15 seconds or less, or about 10 seconds or less.

The analyte can be any substance which generates from the composition any compound described herein (e.g., a compound of Formula I, II, IIa, III, IIIa, IlIb, and IV-XV), wherein X is an oxy anion. For example, in various embodiments, the analyte can be an alkaline phosphatase, a peptidase, a glucosidase, an oxidizing agent such as hydrogen peroxide or other reactive oxygen species, glutathione, fluoride, or a base under alkaline conditions.

The present disclosure further provides a kit for determining the presence of an analyte, the kit comprising the compound of any one or more of the compounds described herein, an olefin precursor thereof, a salt thereof, or a composition comprising the same. The kit can contain instructions according to the method described herein.

The compounds and compositions described herein can be triggered directly by an analyte so as to produce a signal identifying the presence of the analyte and probe or can be triggered in a two step-process, one step which involves contacting the analyte and another step which involves raising the pH.

The compounds of the present disclosure can be configured as probes to detect variety of different analytes by modifying group X or G. For example, the compounds described here can be configured to detect β-galactosidase by furnishing a glucosyl group at G, configured to detect hydrogen peroxide or other oxidizing agents by furnishing a boronate at X, configured to detect alkaline phosphatase by furnishing a phosphate at X or a phosphoryl group at G, configured to detect glutathione by furnishing a dinitrobenzenesulfonylaminobenzyl group at G, configured to detect fluorine by furnishing a trialkylsilyl group at G, and configured to detect alkaline conditions when X is OH.

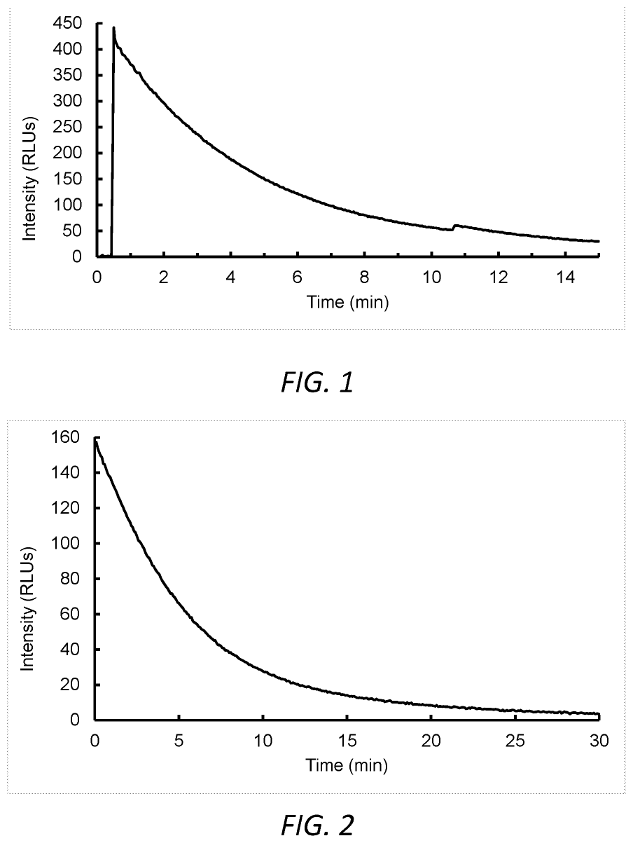

It is desirable that the compounds emit all possible light in the briefest possible period of time upon being triggered by the analyte so as to provide the strongest signal possible. When the chemiluminescence is emitted gradually over a period of time, light intensity (photons/sec) is diminished and detection sensitivity can be impaired. An example of the profile of a luminescent signal of time is shown in FIG. 1.

The rate of luminescence increase, or rise time, can be described according to either the time to the maximum emission (tₘₐₓ) or the emission half-life (T_{1/2}).

The compounds described herein can be used as an enzyme substrate. For example, various compounds in which G is a phosphorous-containing group can be used as a substrate for alkaline phosphatase (ALP) enzyme, and the like. Without being limited by theory, one example mechanism involves the ALP enzyme hydrolyzing the phosphorous-containing group to provide a phenol which is immediately deprotonated due to the alkaline environment of the solution (e.g., pH 9.7 buffer). Formation of the oxy anion trigger decomposition of the 1,2-dioxetane into two compounds: 2-adamantanone and a phenyl ester that is at an excited state. The excited phenyl ester then immediately decays to the ground state by releasing light.

The resulting light intensity is a linear function of the amount of the enzyme. The compounds described herein can thus be used to detect a label enzyme used in an assay. For example, the steps of the chemical process in which the dioxetane provides light can be described according to the following steps: (i) X+S→X+S′(ii) S′→P* and (iii) P*→P+light. Step (i) represents catalytic turnover of the substrate, wherein X is an enzyme or other component that converts the substrate to its activated form, step (ii) represents degradation of the activated substrate to a transient excited species, and step (iii) represents decay of the excited species to ground state and emission of light. Light intensity is the product of the catalytic turnover of substrate in step (i) and the lifetime of the resulting light-producing compound P* in step (ii). Step (ii) is usually first order with a rate constant k and can be characterized by its half-life: T_{1/2}=(ln 2)/k. Step (iii) is extremely short in comparison to the other steps and generally has no meaningful effect on reaction kinetics.

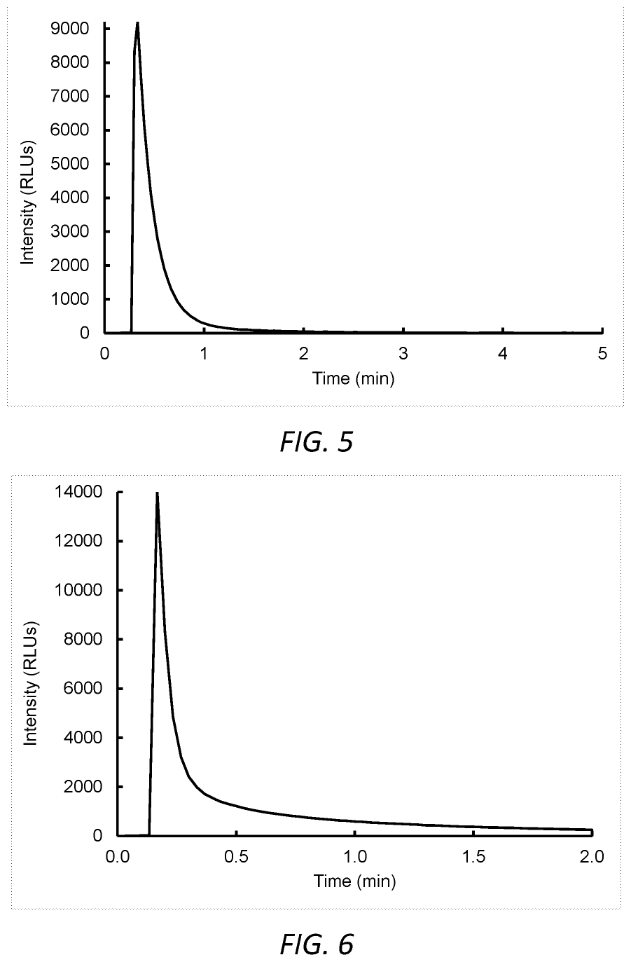

Chemiluminescence intensity/time profile comprises a period of initial rising emission intensity and a subsequent period of steady-state intensity. A slow first order reaction of S′→P* corresponds to an extended rise time as it takes longer for the steady state concentration of S′ to be reached. Fast reaction of S′→P* corresponds to shorter initial rising period and thus provides a rapid rise. For enzymatic chemiluminescent reactions, intensity will typically remain plateaued at a high level and the resulting signal will typical correspond to the shape of the signal shown in FIG. 5. The absence of a steady intensity indicates either substrate depletion or subsequent inactivation of the enzyme. Detection of enzyme-generated chemiluminescence provides flexibility in the measurement process as light intensity at any time point can be related to the amount of enzyme, however enzyme generated processes can have disadvantages, e.g., due to the size and “sticky” nature of the enzyme label. However, for maximal sensitivity measurement is optimally conducted at or near the maximum intensity (Iₘₐₓ) during the period of steady-state intensity.

The compounds described herein can also be used as a direct label for one of the complementary binding partners in an immunoassay. The compounds described herein are advantageously used as labels because they are small molecules, in contrast to large bioluminescent molecules and other types of enzyme labels.

The present disclosure thus also relates to an assay that uses the compounds described herein as a chemiluminescent probe.

In various embodiments, the assay can be a homogeneous (non-separation) assay in which bound and unbound ligands do not need to be separated, or the assay can be a heterogeneous assay in which labeled binding pair complexes are separated from unbound labeled reactants. The assay can be configured to be performed manually or it can be automated and performed robotically. The assay can be performed in test tubes, cuvettes, microwells, or a combination thereof. In various embodiments, the test tubes, cuvettes, microwell, or other containers in which the assay is performed are at least partially opaque, fully opaque, black, white, or a combination thereof.

The assay can be performed on immobilized proteins in western blots, immobilized nucleic acids in Southern or northern blots.

Imaging can be recorded using a luminometer, x-ray film, or a charge coupling device (CCD) camera system.

Measuring chemiluminescence has advantages over fluorescence and absorption spectroscopy. For example, fluorescence and absorption spectroscopy can suffer from interfering signals produced from either the incident light or background signals.

The assays described herein can be configured to measure chemiluminescence according to the non-limiting examples described in J. E. Wampler, Instrumentation: Seeing the Light and Measuring It, in Chemi- and Bioluminescence, J. G. Burr, ed., Marcel Dekker, New York, 1-44 (1985), A. K. Campbell, Detection and Quantification of Chemiluminescence, in Chemiluminescence Principles and Applications in Biology and Medicine, Ellis Horwood, Chichester, 68-126 (1988), F. Berthold, Instrumentation for Chem iluminescence Immunoassays, in Luminescence Immunoassay and Molecular Applications, K. Van Dyke and R. Van Dyke, eds., CRC Press, Boca Raton, 11-25 (1990), and T. Nieman, Chemiluminescence: Theory and Instrumentation, Overview, in Encyclopedia of Analytical Science, Academic Press, Orlando, 608-613 (1995), each of which is incorporated by reference herewith in its entirety.

A number of approaches can be used to attach the compounds of the present disclosure to a biological molecule. For example, where the compound contains a reactive group such as, for example, carboxyl, carboxyl halide, sulfonyl halide, carboalkoxy, carboxamido, carboxime, or N-succinimidylcarboxy, such groups can be coupled covalently to hydroxyl functions or amino functions using conjugation reagents such as, for example, carbodiimides or 1,1-carbonyldiimidazole. N-maleimido groups react directly with sulfhydryl residues in proteins. If the compound contains aromatic amino groups, these can be converted to diazonium salts and reacted with phenol groups such as those found in tyrosine groups of proteins. Either a reactive group present in the polycyclic aromatic moiety or other light producing group, or one present in the leaving group, can be used to attach the compounds of the present invention to the biological molecule.

Compounds of the present disclosure can generally be prepared, for example, according to the synthetic approach described in Scheme 1.

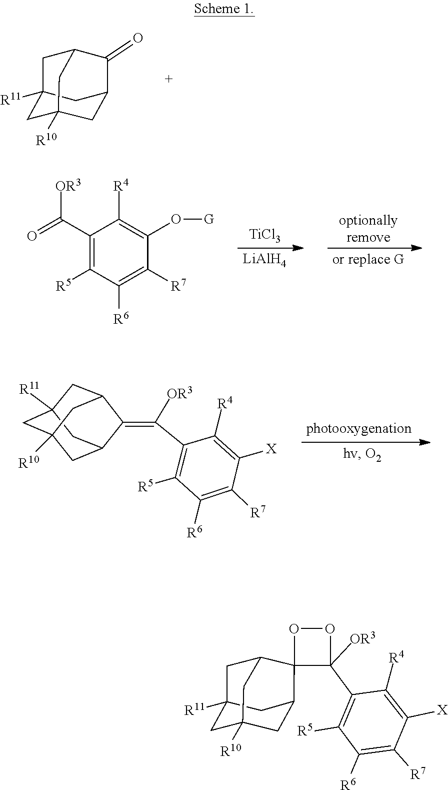

A 2-adamantanone and a 3-substituted benzoate ester can be coupled together by subjecting them to McMurry reaction conditions involving oxophilic titanium and a reducing agent. The resulting olefin can be further modified, e.g., via removing or replacing protecting group G or further functionalizing positions R⁴, R⁵, R⁶, and R⁷ on the ring. Next, the olefin is subjected to photooxygenation conditions to provide a 1,2-dioxetane product. In various embodiments, R³, R⁴, R⁵, R⁶, R⁷, R¹⁰, R¹¹, G, and X are as described in any of the various embodiments of this application, e.g. as described according to Example 1. In some embodiments, R⁴, R⁵, R⁶, R¹⁰, and R¹¹ are H. In some embodiments, R³ is substituted or unsubstituted alkyl and R⁷ is an electron donating group.

The term light “intensity” or luminescence “intensity” as used herein refers to the rate of emission in photons/sec. Intensity can be measured by use of a luminometer. A luminometer is a photodetector in a housing which excludes ambient light. Any suitable luminometer can be used, including photomultiplier tubes and photodiodes.

The term “speed of luminescence” refers to the rate of luminescence increase, i.e., the change in light intensity over time.

The term “sensitivity” as used herein refers to the lowest level at which a signal for an analyte or product being measured can be reproducibly detected.

The term “alkyl” as used herein refers to substituted or unsubstituted straight chain, branched or cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, bicyclo[2.2.1]heptyl, and adamantyl. Cycloalkyl groups further include substituted and unsubstituted polycyclic cycloalkyl groups such as, but not limited to, norbornyl, adamantyl, bornyl, camphenyl, isocamphenyl, and carenyl groups, and fused rings such as, but not limited to, decalinyl, and the like. For example cycloalkyl includes an adamantyl substituted by one, two, three, four, or more substituents, e.g., at the tertiary bridgehead positions at the methylene bridges. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl.

The term “alkenyl” as used herein refers to substituted or unsubstituted straight chain, branched or cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Where specified, the parent moiety should be understood to be attached to the alkenyl group at a vinylic position of the double bond rather than a non-vinylic position. For example, where an aromatic ring is substituted with a π-conjugated alkenyl group, it should be understood to be substituted at the vinyl position rather than a non-vinylic position. As a further example, an aromatic ring substituted with a π-conjugated propenyl group would be understood to be a propen-1-yl or a propen-2-yl group rather than a propen-3-yl group. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. The term “heteroaryl” is a fully aromatic heterocyclyl and thus a subset of the term heterocyclyl. The term “heterocycloalkenyl” refers to a heterocyclyl group containing an olefin within a non-aromatic ring, such that the olefin is the point of connection to the parent moiety. A heterocyclyl group can thus be a heterocycloalkyl, heterocycloalkenyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise, a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase heterocyclyl group includes fused ring species including those that include fused aromatic and non-aromatic groups. Representative heterocyclyl groups include, but are not limited to piperidynyl, pyrrolidinyl, piperazinyl, and morpholinyl. For example, heterocyclyl groups include, without limitation:

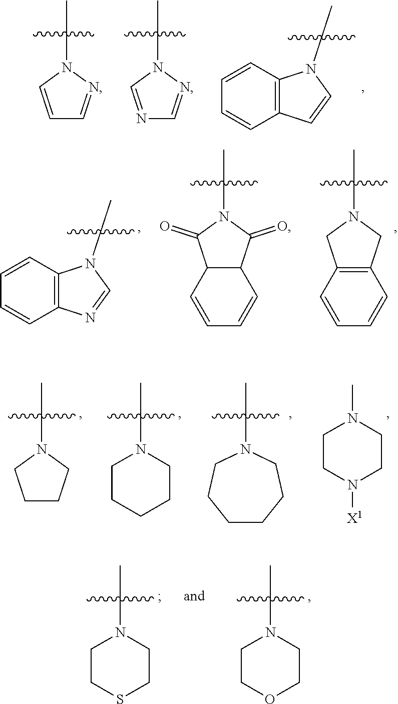

wherein X¹ represents H, (C₁-C₂₀)alkyl, (C₆-C₂₀)aryl or an amine protecting group (e.g., a t-butyloxycarbonyl group) and wherein the heterocyclyl group can be substituted or unsubstituted. Representative heteroaryl groups include furanyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. In some embodiments, the heteroaryl is a 5-membered heteroaryl. In some embodiments, the heteroaryl is other than pyridine, pyrimidine, pyridazine, pyrazine, or fused derivatives thereof. A π-excessive heteroaryl is a heteroaryl that is electron-rich such that it can function as an electron donating group. Examples of π-excessive heteroaryls are furan, thiophene, indole, pyrrole, benzofuran, and benzothiophene.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein. The point of substitution to the parent moiety is at the oxygen atom.

The term “arylcarbonyl” as used herein refers to a carbonyl (CO) group connected to an aryl group as are defined herein. The point of substitution to the parent moiety is at the carbonyl group.

The term “heteroarylcarbonyl” as used herein refers to a carbonyl (CO) group connected to an heteroaryl group as are defined herein. The point of substitution to the parent moiety is at the carbonyl group.

The term and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkenyl group is replaced with a bond to an aryl group as defined herein. The point of substitution to the parent moiety is at the alkyl group.

The terms “halo,” “halogen,” or “halide” group, as used herein, by themselves or as part of another substituent, mean, unless otherwise stated, a fluorine, chlorine, bromine, or iodine atom.

The term “amino” as used herein refers to a substituent of the form —NH₂, —NHR, —NR₂, —NR₃+, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, alkoxy, aryl, cycloalkyl, heterocyclyl, group or the like.

The term “formyl” as used herein refers to a group containing an aldehyde moiety. The point of substitution to the parent moiety is at the carbonyl group.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to a nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “alkylthio” as used herein refers to a sulfur atom connected to an alkyl, alkenyl, or alkynyl group as defined herein. The point of substitution to the parent moiety is at the sulfur atom.

The term “arylthio” as used herein refers to a sulfur atom connected to an aryl group as defined herein. The point of substitution to the parent moiety is at the sulfur atom.

The term “alkylsulfonyl” as used herein refers to a sulfonyl group connected to an alkyl, alkenyl, or alkynyl group as defined herein. The point of substitution to the parent moiety is at the sulfonyl group.

The term “alkylsulfinyl” as used herein refers to a sulfinyl group connected to an alkyl, alkenyl, or alkynyl group as defined herein. The point of substitution to the parent moiety is at the sulfinyl group.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups. The point of substitution to the parent moiety is at the sulfonyl group.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups. The point of substitution to the parent moiety is at the nitrogen atom.

The term “dialkylamido” as used herein refers to an amido group connected to two alkyl groups, as defined herein, and which can optionally be linked together to form a ring with the nitrogen. This term also includes the group where the nitrogen is further connected to one or two alkenyl groups in place of the alkyl groups. The point of substitution to the parent moiety is at the amido group.

Each of the various substituent groups described herein can be substituted or unsubstituted. The term “substituted” as used herein refers to a group that is substituted with one or more groups (substituents) including, but not limited to, the following groups: deuterium (D), halogen (e.g., F, Cl, Br, and I), R, OR, OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl or (C₆-C₂₀)aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups. In various embodiments, a substituted group may be substituted with a group other than a carbonyl-containing group, nitro, cyano, sulfinyl, sulfonyl, or a halogen-containing group. In various embodiments, a substituted group may be substituted with a group other than an electron-withdrawing group. Some substituted groups in certain embodiments may be substituted solely with one or more electron-donating groups.

The term “boronate group” as used herein refers to the following structure in which R⁸ and R⁹ are each independently H or C₁-C₁₀ alkyl, or R⁸ and R⁹ taken together with the boronate to which they are attached provide a C₂-C₁₀ cyclic boronate ester

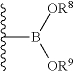

As used herein, the term “π-conjugated” group refers to a substituent that has an unhybridized P-orbital that overlaps or aligns with an unhybridized P-orbital in the parent moiety to which the π-conjugated is attached, such that electrons may be shared between the two P-orbitals and a lower energy state is achieved. An example parent moiety is the phenyl group to which X, R⁴, R⁵, R⁶, and R⁷ are attached. A substituent that is a π-conjugated group can also have r-bonding electrons that are delocalized through both the substituent and the parent moiety to which it is attached. Examples of π-conjugated groups include substituted or unsubstituted C₂-C₁₀ alken-1-yl, C₂-C₁₀ alken-2-yl, C₂-C₁₀ alkenyn-1-yl, C₂-C₁₀ heterocycloalken-1-yl, C₂-C₁₀ heterocycloalken-2-yl, C₆-C₁₀ aryl, or heteroaryl. Further examples of π-conjugated groups include substituted or unsubstituted vinyl, ethynyl, C₆-C₁₀ aryl, or heteroaryl further substituted with a C₂-C₁₀ alken-1-yl, C₂-C₁₀ alken-2-yl, C₂-C₁₀ alkenyn-1-yl, C₂-C₁₀ heterocycloalken-1-yl, C₂-C₁₀ heterocycloalken-2-yl, C₆-C₁₀ aryl, or heteroaryl. Yet further examples include, for example, substituted or unsubstituted biaryl, biheteroaryl arylvinyl, heteroaryl vinyl, and C₂-C₁₀ alken-1-yl aryl, phenyl heteroaryl, and heteroaryl aryl.

As used herein, the term “electron-donating group” refers to a group that has a net electron donating effect relative to hydrogen. Electron-donating groups are well known in the art. See, for example, Jerry March, Michael B. Smith, March's Advanced Organic Chemistry 6th edition, 2007, Wiley Interscience and J. McMurry, Organic Chemistry, 5th Ed. (Brooks/Cole, Pacific Grove, 2000), each of which are incorporated by reference herewith in their entireties. Electron-donating groups, sometimes abbreviated EDGs, can be defined according to their Hammett Substituent Constant also known as sigma values (a values). In various embodiments, the electron donating group has a sigma value of 0.3 or lower, 0.2 or lower, 0.1 or lower, or a negative sigma value. In further embodiments, embodiments, the electron donating group is a non-halogen group having a has a sigma value of 0.3 or lower, 0.2 or lower, 0.1 or lower, or a negative sigma value. In cases where the position of the substituent substantially influences its sigma value, the sigma value should be determined relative to the position of the group X. For example, σₘₑₜₐ values could be provided to determine the sigma value of a substient at R⁶ and σₚₐᵣₐ values could be provided to determine the sigma value of a substituent at R⁵. Sigma values can be obtained according to published tables or experimentally. See, for example, J. E. Leffler and E. Grunwald, Rates and Equilibria of Organic Reactions, Wiley, 1963 (Dover reprint), which is incorporated by reference herewith in its entity. Various examples of electron donating groups include oxy anion, hydroxyl, amino, thio. alkylamino, dialkylamino, alkoxy, alkylthio, acylamino, acyloxy, alkyl, alkenyl, vinyl, aryl, electron-rich heteroaryl,

A further manner of determining whether a particular substituent on a given structure is electron donating is by comparing the pKa of the phenolic group (i.e., X═OH) of the substituted structure with the pKa of the phenolic of an unsubstituted but otherwise identical structure. For example, one could compare the phenol pKa values for a compound where R⁴, R⁵, and R⁶ are H and R⁷ is vinyl to a compound where R⁴, R⁵, R⁶, and R⁷ are H.

In various embodiments, R⁴, R⁵, R⁶, and R⁷ can be understood to provide a net donating effect if the pKa of a phenolic group at X is 9.0 or greater, 9.5 or greater, 10.0 or greater, 10.5 or greater, or 11.0 or greater. In various embodiments, R⁴, R⁵, R⁶, and R⁷ provide a net electron donating effect if the pKa of a phenolic group at X is greater than that of a compound where R⁴, R⁵, R⁶, and R⁷ are H.

In further embodiments, X is OH or an oxy anion and has a pKa of 9.0 or greater, 9.5 or greater, 10.0 or greater, 10.5 or greater, or 11.0 or greater.

An “electron-withdrawing group,” sometimes abbreviated EWG, refers to a group that has a net electron withdrawing effect relative to hydrogen. Electron-withdrawing groups are well known in the art. See, for example, Jerry March, Michael B. Smith, March's Advanced Organic Chemistry 6th edition, 2007, Wiley Interscience and J. McMurry, Organic Chemistry, 5th Ed. (Brooks/Cole, Pacific Grove, 2000), each of which are incorporated by reference herewith in their entireties. Although it is thought that the presence of an EWG slows the rate of formation of the reactive luminescent intermediate, some embodiments of the present disclosure can contain one or more EWG, provided that the net overall effect of substituents is an electron donating effect. For example, in some embodiments, R⁴, R⁵, R⁶, and R⁷ can include one or more electron-withdrawing group (EWG) provided that R⁴, R⁵, R⁶, and R⁷ have an overall net electron donating effect on the aryl ring to which they are attached. Examples of electron withdrawing groups include acrylate groups, such as alkyl acrylate (e.g., CH₃C(O)CH═CH—) and cyanoacrylate (NCCH═CH—) groups.

As used herein, the term alcohol protecting group refers to a substituent group on an oxy group which renders the oxygen inert to various conditions in which an alcohol would typically react, but which is readily removed when subjected to certain conditions. Alcohol protecting groups as described herein will typically improve the stability of the dioxetane moiety, and upon their removal will promote decomposition of the dioxetane. Thus, alcohol protecting groups include phosphates such as PO₃Na₂, PO₃Cl₂, and PO₃H₂, glycosyl groups, dinitrobenzenesulfonylaminobenzyl groups, and other groups which can be enzymatically hydrolyzed to provide the unprotected alcohol. Some alcohol protecting groups are described in Theodora W. Greene, Peter G. M. Wuts (1999). Protecting Groups in Organic Synthesis (3 ed.). J. Wiley, which is incorporated herewith in its entirety. Alcohol protecting groups include acetyl, benzoyl, benzyl, methoxyethoxymethyl, dimethyltrityl, methoxylmethyl, methylthiomethyl, pivaloyl, tetrahydropyranyl, tetrahydrofuranyl, trityl, trialkylsilyl, trialkylsiloxymethyl, dialkylarylsilyl, glycosyl, pyranyl, galactosyl, and ethoxyethyl groups. Alcohol protecting groups also include groups in which the alcohol is substituted with a fragmentable linker that is further substituted with a protecting group, wherein upon deprotecting of such protecting group the linker fragments and eliminates from the alcohol. The following compounds are yet further examples of alcohols substituted with an alcohol protecting group:

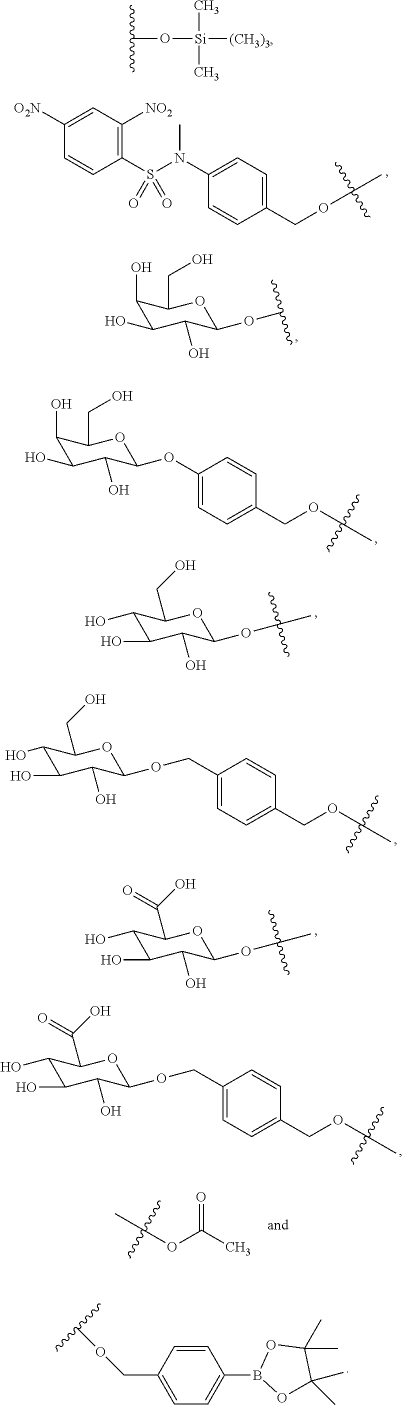

In various embodiments, the protecting group G may be an enzyme-cleavable group, wherein removal of said cleavable group by the analyte of interest, e.g., in the presence of an enzyme capable of cleaving said enzyme cleavable group, provides the unstable phenolate-dioxetane species that subsequently decomposes and emits light. For example, G may be a peptide moiety consisting of two or more amino acid residues cleavable by a specific enzyme.

As used herein, the term “surfactant-based luminescence enhancer” refers to the type of compounds typically used to increase the intensity of dioxetanes in aqueous solutions. Emerald™ and Emerald-II™ enhancers are examples of surfactant-based dyes that are commercially available from Thermo Fisher Scientific (Waltham, Mass.). Further examples of surfactant-based luminescence enhancers are described in Schaap, A. P.; Akhavan, H.; Romano, L. J. Clin. Chem. 1989, 35(9), 1863, which is incorporated by reference in its entirety. In various embodiments, the surfactant-based luminescence enhancer contains a tail portion, which is an acyclic alkyl group of at least 8 carbons, and a head portion, which is one or more quaternary ammonium salt, pyridinium salt, quaternary phosphonium surfactant salt, ethyleneglycol chain, or fluorescein moiety. In various embodiments, the surfactant-based luminescence enhancer is a cationic surfactant-based luminescence enhancer such as cetrimonium bromide (CTAB), cetylpyridinium chloride (CPC), benzalkonium chloride (BAC), benzethonium chloride (BZT), dimethyldioctadecylammonium chloride, dioctadecyldimethylammonium bromide (DODAB), α′-tributylphosphonium-p-xylene dichloride, poly(vinylbenzyl tributylphosphonium chloride) (TBE), poly(vinylbenzyl trioctylphosphonium chloride), and the like. In various further embodiments, the surfactant-based luminescence enhancer is a non-ionic Triton X-100, Tween surfactants, surfactants having a long alkyl chain having a polyethyleneglycol head, Brij® surfactants, IGEPAL® surfactants, octylphenoxypolyethoxyethanol, and the like. The surfactant-based luminescence enhancer can also include surfactants having a fluorescent head groups, such as N-(3′,6′-dihydroxy-3-oxo-3H-spiro[isobenzofuran-1,9′-xanthen]-5-yl)tetradecanamide (fluorescein surfactant), and the like.

In some instances, the compounds described herein (e.g., the compounds of the Formulae (I)-(X) can contain chiral centers. All diastereomers of the compounds described herein are contemplated herein, as well as racemates.

As used herein, the term “salts” and “pharmaceutically acceptable salts” refer to derivatives of the disclosed compounds wherein the parent compound is modified by making acid or base salts thereof. Examples of salts include alkali salts and alkali earth salts of an ionized form of the disclosed compounds. For example, a lithium salt, sodium salt, potassium salt, calcium salt, or magnesium salt. The disclosed compounds may be a salt comprising a cationic metal and an anionic organic compound, for example, a compound having an oxyanion and a sodium cation. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Each embodiment described above is envisaged to be applicable in each combination with other embodiments described herein. For example, embodiments corresponding to Formula (I) are equally envisaged as being applicable to Formulae (II)-(X). As another example, embodiments corresponding to Formula (II) are equally envisaged as being applicable to each of Formulae (I) and (III)-(X).

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” or “substantially free of” as used herein refers to less than about 1%, 0.5%, 0.1%, 0.05%, 0.001%, or at less than about 0.0005% or less, about 0%, below quantitation limits, below detectable limits, or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The present invention can be better understood by reference to the following examples which are offered by way of illustration. The present invention is not limited to the examples given herein.

### General Methods

Various compounds of the present disclosure can be synthesized according to various methods including, but not limited to, the synthetic approaches described in PCT International Application WO1996/015122 A1, U.S. Pat. No. 4,962,192, or U.S. Pat. No. 5,004,565, each of which is incorporated by reference herewith in their entireties.

Chemiluminescence (emission) intensity can be measured using a Turner Designs (Sunnyvale, Calif.) model TD-20e luminometer, a BMG Labtech luminescence plate reader, or a charge-coupled device (CCD) camera luminometer, or any other suitable light intensity measuring devices. In the examples listed below, solutions containing alkaline phosphatase (e.g., AP4, AP6, AP8 and AP9) at different concentrations were used, such as, with each number presenting 104, 10⁶, 10⁸, and 109 serial dilution from the initial stock. The compounds and enhancers (if used) were tested at their approximately optimal concentrations.

Nuclear magnetic resonance (NMR) spectra were obtained using a 400 MHz spectrometer in solutions of D₂O and CDCl₃.

The amine-based buffer “221” or “Sigma-221” can be obtained from Sigma-Aldrich (St. Louis, Mo.).

### Example 1

A dioxetane compound was obtained having the following structure:

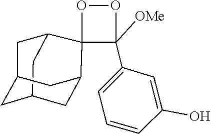 [SMILES: COC1(c2cccc(O)c2)OOC12C1CC3CC(C1)CC2C3]

¹H NMR (400 MHz, CDCl₃) δ ppm 7.30-7.05 (m, 3H), 6.90 (m, 1H), 6.28 (br, 1H), 3.22 (s, 3H), 3.04 (s, 1H), 2.21 (s, 1H), 1.94-1.6 (m, 10H), 1.24 (m, 1H), 1.04 (m, 1H).

An initial solution of the compound was prepared in dioxane (1 mg compound per 1 mL dioxane), which was then mixed with water (20 μL of dioxane solution in 180 μL water) and subsequently treated with 200 μL of amine-based 221 buffer at 37° C. Intensity of chemiluminescence was measured over time upon treating the compound with the alkaline buffer solution. A graph showing the intensity of light emission over time is provided at FIG. 1 showing that light emission is drawn out over a long period of time. The compound of Example 1 shows a chemiluminescence half-life of 3.79 minutes and a Σ value of 1.13E+5.

### Example 2

A dioxetane compound was obtained having the following structure:

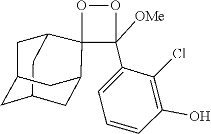 [SMILES: COC1(c2cccc(O)c2Cl)OOC12C1CC3CC(C1)CC2C3]

¹H NMR (400 MHz, CDCl₃) δ ppm 7.63 (m, 1H), 7.31 (m, 1H), 7.15 (m, 1H), 6.0 (br, 1H), 3.22 (s, 3H), 3.0 (s, 1H), 2.24 (s, 1H), 2.1-1.4 (m, 12H).

Example 2 was tested in a manner analogous to Example 1, using 10 μL of a 1 mg/mL sample of the test compound in THF. The compound of Example 2 shows a chemiluminescence half-life of 4.91 minutes. Addition of the electron-withdrawing chlorine on the phenyl ring at the ortho position results in a slower rate increase of emission and a longer half-life for light emission.

### Example 3

A dioxetane compound was obtained having the following structure:

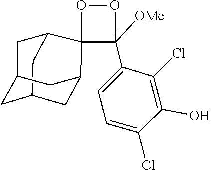 [SMILES: COC1(c2ccc(Cl)c(O)c2Cl)OOC12C1CC3CC(C1)CC2C3]

¹H NMR (400 MHz, CDCl₃) δ ppm 7.05 (m, 2H), 3.44 (s, 3H), 3.24 (s, 1H), 2.54 (s, 1H), 2.1-1.6 (m, 12H).

Example 3 was tested in a manner analogous to Example 2. The compound of Example 3 shows a chemiluminescence half-life of 9.22 minutes. Addition of two electron-withdrawing chlorine groups results in an even slower rate increase of emission and a longer half-life for light emission.

### Example 4

A dioxetane compound was obtained having the following structure:

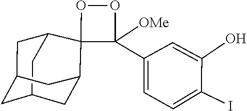 [SMILES: COC1(c2ccc(I)c(O)c2)OOC12C1CC3CC(C1)CC2C3]

¹H NMR (400 MHz, CDCl₃) δ ppm 7.70 (m, 1H), 7.45-6.65 (m, 2H), 5.59 (br, 1H), 3.20 (s, 3H), 3.00 (s, 1H), 2.16 (s, 1H), 1.90-1.40 (m, 10H), 1.27 (m, 1H), 1.04 (m, 1).

Example 4 was tested in a manner analogous to Example 2. The compound of Example 4 shows a chemiluminescence half-life of 2.10 minutes. Addition of a slightly electron-donating iodine atom results in a slightly increase rate increase of emission and a slightly shorter half-life compared to Examples 1-3.

### Example 5

A dioxetane compound was obtained having the following structure:

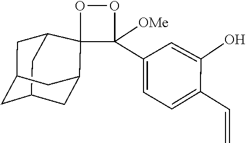 [SMILES: C=Cc1ccc(C2(OC)OOC23C2CC4CC(C2)CC3C4)cc1O]

¹H NMR (400 MHz, CDCl₃) δ ppm 7.44 (m, 1H), 7.40-6.90 (m, 3H), 5.82 (d, J=16 Hz, 1H), 5.65 (br, 1H), 5.41 (d, J=16 Hz, 1H), 3.23 (s, 3H), 3.02 (s, 1H), 2.18 (s, 1H), 1.90-1.40 (m, 10H), 1.25 (m, 1H), 1.09 (m, 1H).

Example 5 was tested in a manner analogous to Example 1, using 10 μL of a 0.1 mg/mL sample of the test compound in dioxane. Specifically, an initial solution of the compound was prepared in dioxane (0.1 mg compound per 1 mL dioxane), which was then mixed with water (10 μL of dioxane solution in 100 μL water) and subsequently treated with 200 μL of amine-based 221 buffer at 37° C.

A graph showing the intensity of light emission over time is provided at FIG. 5. The compound of Example 5 showed a sharp signal and a chemiluminescence half-life of 23 seconds and a Σ value of 1.28E+5. Addition of an electron donating vinyl group on the phenyl ring of PPD resulted in a higher intensity of emission, a more quickly increasing rate of emission, and a dramatically shorter half-life for light emission compared to Examples 1-4.

### Example 6

A dioxetane compound was obtained having the following structure:

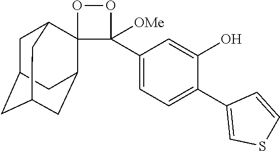 [SMILES: COC1(c2ccc(-c3ccsc3)c(O)c2)OOC12C1CC3CC(C1)CC2C3]

¹H NMR (400 MHz, CDCl₃) δ ppm 7.51 (m, 1H), 7.48 (m, 1H), 7.41 (m, 1H), 7.35 (m, 1H), 7.40-7.01 (m, 2H), 5.52 (br, 1H), 3.26 (s, 3H), 3.04 (s, 1H), 2.26 (s, 1H), 1.90-1.46 (m, 10H), 1.28 (m, 1H), 1.13 (m, 1H).

Example 6 was tested in a manner analogous to Example 1. The compound of Example 6 showed a chemiluminescence half-life of 12.7 seconds. Addition of an even more electron donating electron donating 3-thienyl group resulted in a higher intensity of emission, a more quickly increasing rate of emission, and a shorter half-life for light emission compared to Examples 1-5.

### Example 7

A dioxetane compound was obtained having the following structure:

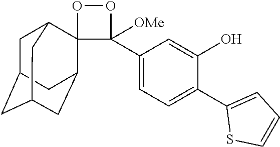 [SMILES: COC1(c2ccc(-c3cccs3)c(O)c2)OOC12C1CC3CC(C1)CC2C3]

¹H NMR (400 MHz, CDCl₃) δ ppm 7.51 (m, 1H), 7.42 (m, 1H), 7.45-7.01 (m, 4H), 5.80 (br, 1H), 3.26 (s, 3H), 3.04 (s, 1H), 2.08 (s, 1H), 2.1-1.5 (m, 10H), 1.25 (m, 1H), 1.10 (m, 1H).

Example 7 was tested in a manner analogous to Example 1, using 10 μL of a 1 mg/mL sample of the test compound in dioxane. Specifically, an initial solution of the compound was prepared in dioxane (1 mg compound per 1 mL dioxane), which was then mixed with water (10 μL of dioxane solution in 90 μL water) and subsequently treated with 200 μL of amine-based 221 buffer at 37° C.

A graph showing the intensity of light emission over time is provided at FIG. 6. The compound of Example 7 showed a sharp signal and a chemiluminescence half-life of 11.8 seconds and a Σ value of 1.32E+05. Here, the 2-thienyl moved the electron-rich sulfur atom closer to the phenyl ring and thus provided a stronger electron donating effect. The compound of Example 7 resulted in a higher intensity of emission, a more quickly increasing rate of emission, and a shorter half-life for light emission compared to Examples 1-6.

### Example 8

A dioxetane compound was obtained having the following structure:

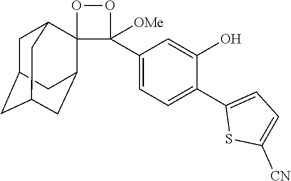 [SMILES: COC1(c2ccc(-c3ccc(C#N)s3)c(O)c2)OOC12C1CC3CC(C1)CC2C3]

¹H NMR (400 MHz, CDCl₃) δ ppm 7.68 (d, J=2 Hz, 1H), 7.62 (d, J=1.2 Hz, 1H), 7.56 (s, 1H), 7.50-7.10 (br, 2H), 6.80 (br, 1H), 3.27 (s, 3H), 3.05 (s, 1H), 2.23 (s, 1H), 1.88-1.45 (m, 10H), 1.31-1.26 (m, 1H), 1.12-1.08 (m, 1H).

Example 8 was tested in a manner analogous to Example 1 using 10 μL of a 0.001 mg/mL sample of the test compound in dioxane. Specifically, an initial solution of the compound was prepared in dioxane (0.001 mg compound per 1 mL dioxane), which was then mixed with water (10 μL of dioxane solution in 90 μL water) and subsequently treated with 100 μL of amine-based 221 buffer at 37° C.

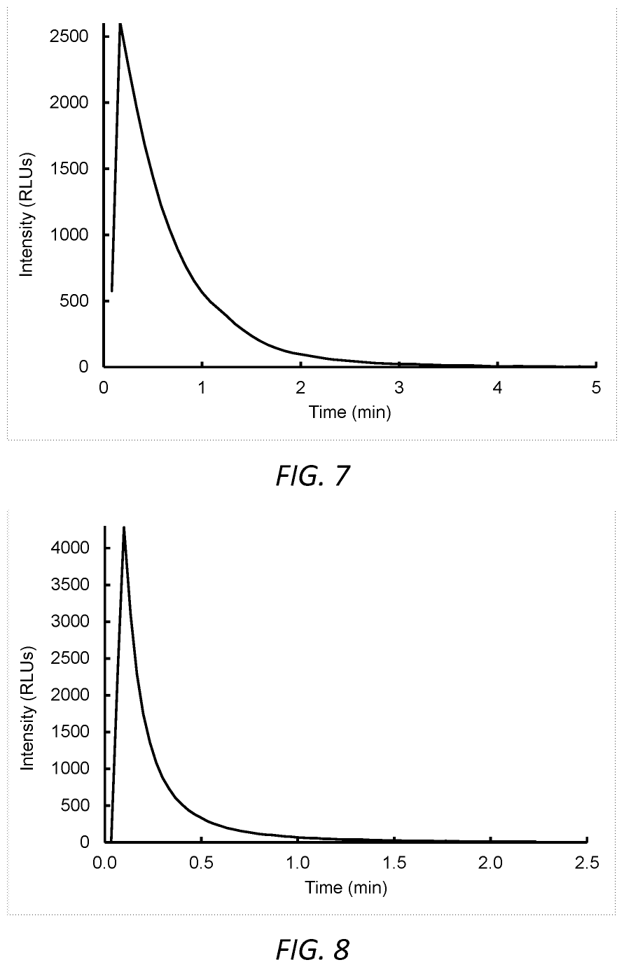

A graph showing the intensity of light emission over time is provided at FIG. 7. The compound of Example 8 showed a sharp signal and a chemiluminescence half-life of 23 seconds and a Σ value of 9.76E+04. While not wishing to be bound by any specific theory, it is believed that because of the extended π conjugated system, the compound of Example 8 resulted in a higher intensity of emission than example 7. Due to the 4-CN electron-withdrawing effect, the half-life of sample 8 is larger than sample 7.

### Example 9

A dioxetane compound was obtained having the following structure:

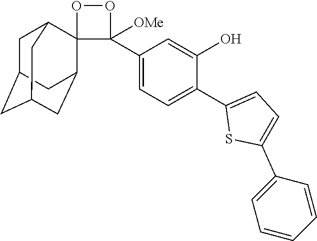 [SMILES: COC1(c2ccc(-c3ccc(-c4ccccc4)s3)c(O)c2)OOC12C1CC3CC(C1)CC2C3]

¹H NMR (400 MHz, CDCl₃) δ ppm 7.72 (d, J=7.2 Hz, 2H), 7.67 (d, J=8 Hz, 2H), 7.60 (d, J=8 Hz, 1H), 7.52 (d, J=4 Hz, 1H), 7.45 (d, J=4 Hz, 1H), 7.44-7.10 (m, 3H, 5.75 (br, 1H), 3.27 (s, 3H), 3.04 (s, 1H), 2.23 (s, 1H), 1.90-1.45 (m, 10H), 1.31-1.26 (m, 1H), 1.15-1.08 (m, 1H).

Example 9 was tested in a manner analogous to Example 1, using 10 μL of a 0.01 mg/mL sample of the test compound in dioxane. Specifically, an initial solution of the compound was prepared in dioxane (0.01 mg compound per 1 mL dioxane), which was then mixed with water (10 μL of dioxane solution in 90 μL water) and subsequently treated with 100 μL of amine-based 221 buffer at 37° C. A graph showing the intensity of light emission over time is provided at FIG. 8.

### Example 10

A dioxetane compound was obtained having the following structure:

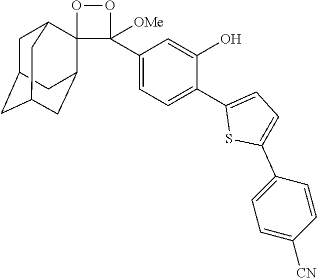 [SMILES: COC1(c2ccc(-c3ccc(-c4ccc(C#N)cc4)s3)c(O)c2)OOC12C1CC3CC(C1)CC2C3]

¹H NMR (400 MHz, CDCl₃) δ ppm 7.72 (d, J=7.2 Hz, 2H), 7.68 (m, 2H), 7.61 (d, J=8 Hz, 1H), 7.52 (d, J=8 Hz, 1H), 7.45 (d, J=4 Hz, 1H), 7.44-7.7.05 (m, 2H), 5.81 (br, 1H), 3.27 (s, 3H), 3.04 (s, 1H), 2.25 (s, 1H), 1.90-1.45 (m, 10H), 1.31-1.26 (m, 1H), 1.15-1.08 (m, 1H).

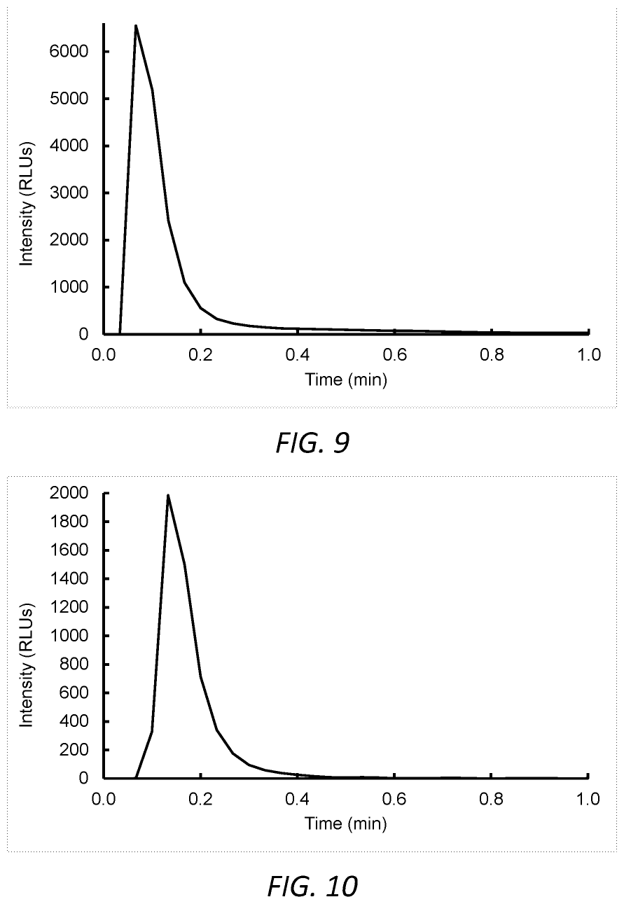

Example 10 was tested in a manner analogous to Example 1, using 10 μL of a 0.01 mg/mL sample of the test compound in dioxane. Specifically, an initial solution of the compound was prepared in dioxane (0.01 mg compound per 1 mL dioxane), which was then mixed with water (10 μL of dioxane solution in 90 μL water) and subsequently treated with 100 μL of amine-based 221 buffer at 37° C. A graph showing the intensity of light emission over time is provided at FIG. 9.

### Example 11

A dioxetane compound was obtained having the following structure:

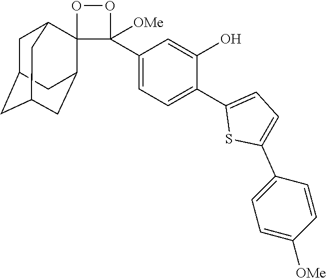 [SMILES: COc1ccc(-c2ccc(-c3ccc(C4(OC)OOC45C4CC6CC(C4)CC5C6)cc3O)s2)cc1]

¹H NMR (400 MHz, CDCl₃) δ ppm 7.56 (m, 2H), 7.42 (m, 1H), 7.44-7.30 (m, 2H), 7.22 (d, J=6 Hz, 2H), 6.93 (d, J=8.8 Hz, 2H), 5.91 (s, 1H), 3.85 (s, 3H), 3.27 (s, 3H), 3.04 (s, 1H), 2.27 (s, 1H), 1.90-1.45 (m, 10H), 1.31-1.26 (m, 1H), 1.15-1.08 (m, 1H).

Example 11 was tested in a manner analogous to Example 1, using 10 μL of a 0.1 mg/mL sample of the test compound in dioxane. Specifically, an initial solution of the compound was prepared in dioxane (0.01 mg compound per 1 mL dioxane), which was then mixed with water (10 μL of dioxane solution in 90 μL water) and subsequently treated with 100 μL of amine-based 221 buffer at 37° C. A graph showing the intensity of light emission overtime is provided at FIG. 10.

### Example 12

A dioxetane compound [Lumigen® PPD] was obtained having the following structure:

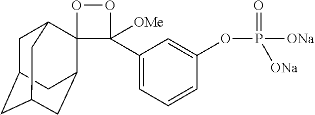

¹H NMR (400 MHz, D₂O) δ ppm 7.40-7.15 (m, 4H), 3.24 (s, 3H), 2.89 (s, 1H), 2.28 (s, 1H), 1.90-1.50 (m, 10H), 1.28 (d, J=13.2 Hz, 1H), 0.99 (d, J=10 Hz, 1H).

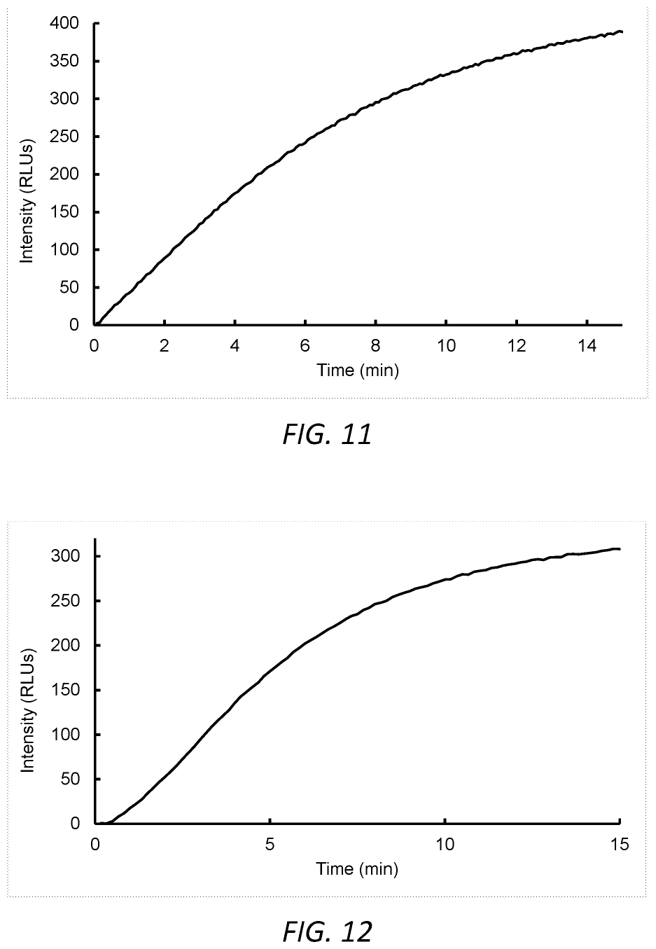

An initial solution of the compound was prepared in 221 buffer (compound: 1.25 mg/ml, TBE enhancer: 5 mg/mL). Next, 100 μL of the initial solution was combined with a solution of 10 μL of an alkaline phosphatase (AP8) at 37° C. The intensity of chemiluminescence was measured over time upon combining the compound with the alkaline phosphatase solution. A graph showing the intensity of light emission over time is provided at FIG. 11. The compound showed a slow and gradual increase in emission intensity which did not reach a maximum light intensity of steady-state plateau for more than 15 minutes and provided a Σ value of 2.27E+05.

### Example 13

A dioxetane compound was obtained having the following structure:

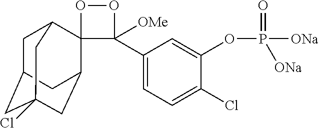

An initial solution of the compound was prepared in 221 buffer (compound: 0.125 mg/mL, TBE enhancer: 2.5 mg/mL), and a 100 μL aliquot was combined with a solution of 10 μL of an alkaline phosphatase (AP8) at 37° C. The intensity of chemiluminescence was measured over time upon combining the compound with the alkaline phosphatase solution. A graph showing the intensity of light emission over time is provided at FIG. 12. The compound showed a slow and gradual increase in emission intensity which did not reach a maximum light intensity of steady-state plateau for more than 15 minutes and provided a Σ value of 1.91E+05.

### Example 14

A dioxetane compound was prepared according to the following structure:

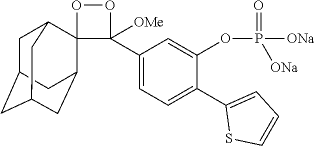

¹H NMR (400 MHz, D₂O) δ ppm 7.86 (br, 1H), 7.81 (d, J=8.4 Hz, 1H), 7.74 (S, 1H), 7.51 (d, J=5.2 Hz, 1H), 7.21 (br, 1H), 7.18 (d, J=7.2 Hz, 1H), 3.28 (s, 3H), 2.90 (s, 1H), 2.33 (s, 1H), 1.85-1.55 (m, 10H), 1.28 (d, J=8 Hz, 1H), 1.10 (d, J=12 Hz, 1H).

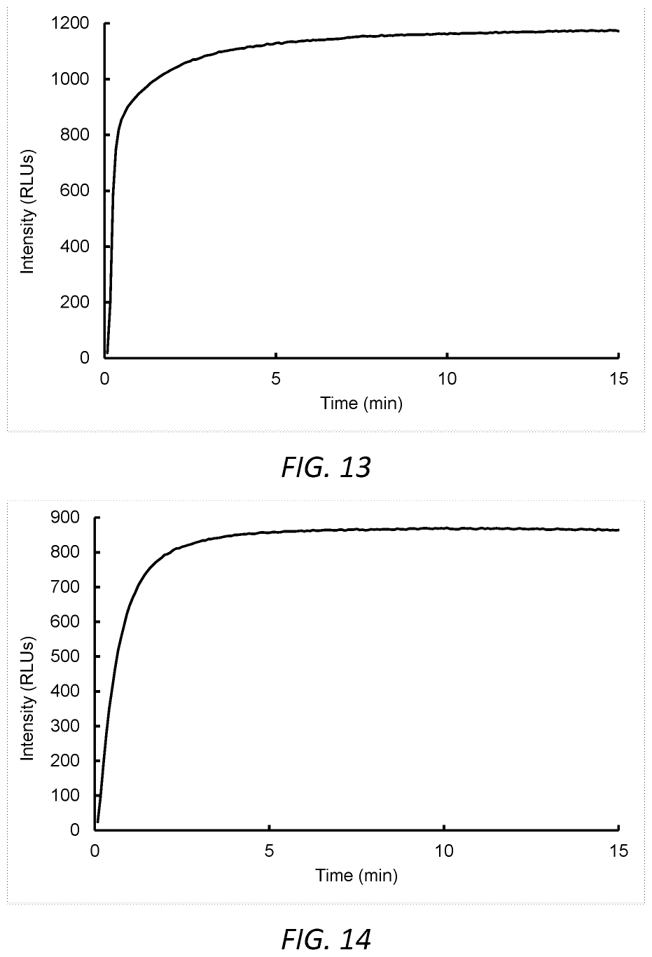

An initial solution of the compound was prepared in 221 buffer (compound: 0.25 mg/mL, TBE enhancer: 5 mg/mL). Next, 100 μL of the initial solution was combined with a solution of 10 μL of an alkaline phosphatase (AP8) at 37° C. The intensity of chemiluminescence was measured over time upon combining the compound with the alkaline phosphatase solution. A graph showing the intensity of light emission over time is provided at FIG. 13. The compound showed a rapid increase in intensity, reached a maximum, steady-state intensity by 1 minute and provided a Σ value of 9.97E+05. The compound of Example 14 provided luminescence having a higher intensity and faster response compared to the compound of Examples 12-13.

### Example 15

A dioxetane compound was prepared according to the following structure:

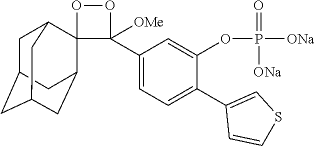

¹H NMR (400 MHz, D₂O) δ ppm 7.95 (m, 1H), 7.85 (br, 1H), 7.65-7.55 (m, 2H), 7.52 (m, 1H), 7.25 (br, 1H), 3.28 (s, 3H), 2.90 (s, 1H), 2.34 (s, 1H), 1.90-1.55 (m, 10H), 1.38 (m, 1H), 1.08 (m, 1H).

An initial solution of the compound was prepared in 221 buffer (compound: 0.25 mg/mL, TBE enhancer: 5 mg/mL). Next, 100 μL of the initial solution was combined with a solution of 10 μL of an alkaline phosphatase (AP8) at 37° C. A graph showing the intensity of light emission over time is provided at FIG. 14. The compound showed a rapid increase in intensity, reached a maximum, steady-state intensity by 2 minute and provided a Σ value of 7.38E+05. The compound of Example 15 provided luminescence having a higher intensity and faster response compared to the compound of Examples 12-13.

### Example 16

A dioxetane compound was prepared according to the following structure:

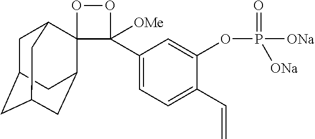

1H NMR (400 MHz, D₂O) δ ppm 7.67 (d, J=8.4 Hz, 1H), 7.66 (br, 1H), 7.26 (br, 1H), 7.16-6.06 (m, 1H), 5.88 (d, J=16 Hz, 1H), 5.37 (d, J=12.4 Hz, 1H), 3.27 (s, 3H), 2.88 (s, 1H), 2.30 (s, 1H), 1.84-1.56 (m, 10H), 1.28 (d, J=9.2 Hz, 1H), 1.05 (d, J=12.8 Hz, 1H).

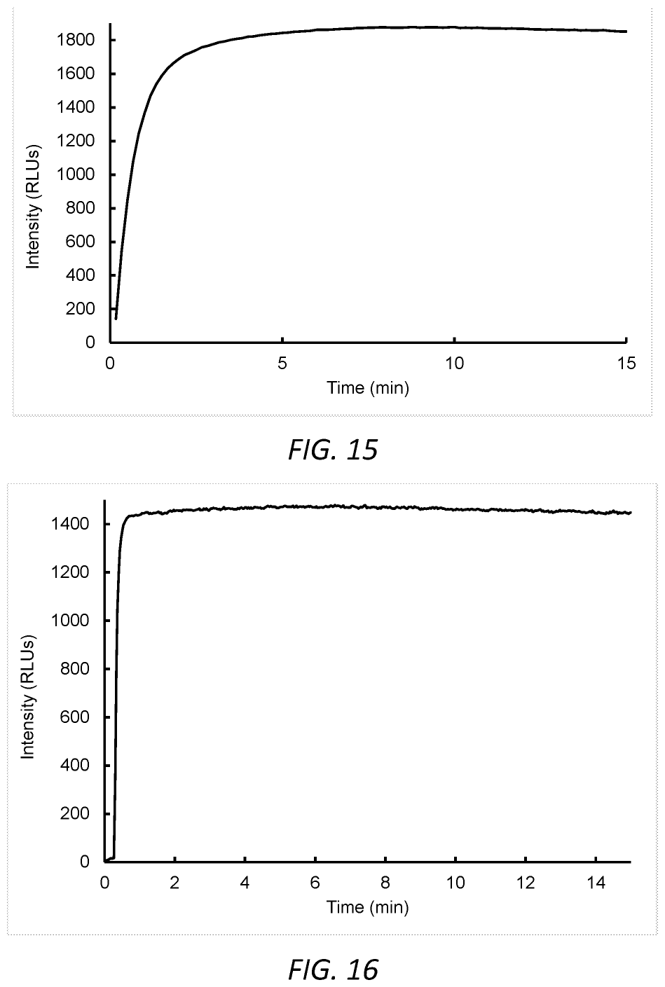

An initial solution of the compound was prepared in 221 buffer (compound: 0.25 mg/mL, TBE enhancer: 2.5 mg/ml). Next, 100 μL of the initial solution was combined with a solution of 10 μL of an alkaline phosphatase (AP8) at 37° C. The intensity of chemiluminescence was measured over time upon combining the compound with the alkaline phosphatase solution. A graph showing the intensity of light emission over time is provided at FIG. 15. The compound showed a rapid increase in intensity, reached a maximum, steady-state intensity in less than 3 minutes and provided a Σ value of 1.59E+06. The compound of Example 16 provided luminescence having a higher intensity and faster response compared to the compound of Examples 12-15.

In a further experiment, an initial solution of the compound was prepared in water (compound: 0.1 mg/mL), Next, 100 μL of the initial solution was combined with a solution of 10 μL of an alkaline phosphatase (AP4) and 300 uL of 5 mg/mL TEB 221 buffer at 37° C. The intensity of chemiluminescence was measured over time upon combining the compound with the alkaline phosphatase solution. A graph showing the intensity of light emission over time is provided at FIG. 16.

### Example 17

A dioxetane compound was prepared according to the following structure:

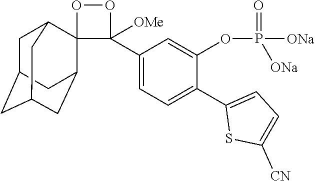

¹H NMR (400 MHz, D₂O) δ ppm 7.88 (m, 2H), 7.75 (m, 1H), 7.52 (m, 1H), 7.30 (br, 1H), 3.26 (s, 3H), 2.88 (s, 1H), 2.29 (s, 1H), 1.90-1.55 (m, 10H), 1.24 (m, 1H), 1.04 (m, 1H).

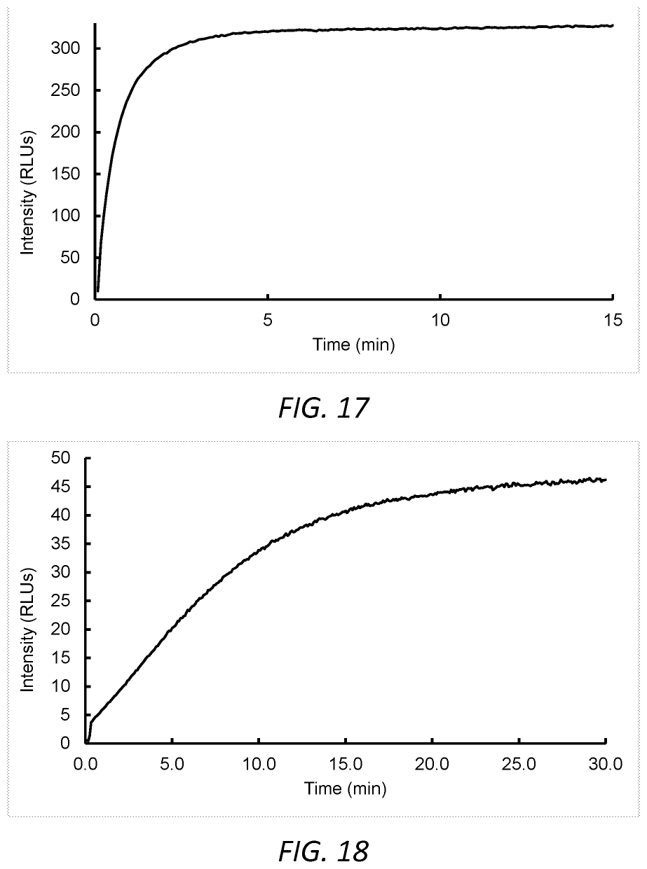

An initial solution of the compound was prepared in 221 buffer (compound: 0.2 mg/mL, without TBE enhancer). Next, 100 μL of the initial solution was combined with a solution of 10 μL of an alkaline phosphatase (AP9, [1.24E-20 mole/μL]) at 37° C. A graph showing the intensity of light emission over time is provided at FIG. 17. The compound showed a rapid increase in intensity, reached a maximum, steady-state intensity by 2 minute and provided a Σ value of 2.77E+05.

FIG. 18 shows the intensity of light emission over time for Lumigen® PPD under similar conditions:

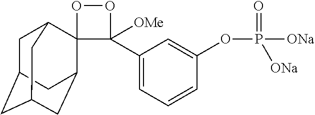

The results show that adding r-conjugation and electron donating groups directly to an aryl ring attached to a dioxetane can result in increased speed and intensity of luminescence upon dioxetane fragmentation (See Examples 5-11 and FIGS. 5-10). Such substitution corresponds, e.g., to placing π-conjugated and electron donating groups at R⁴, R⁵, R⁶, or R⁷ in Formula I or Q in Formula III.

For example, a vinyl substituent resulted in a dioxetane that provided a quick, intense burst of luminescence that was at least 20× more intense than the corresponding unsubstituted compound (See Example 5, compare FIG. 5 and FIG. 1). Substitution with thiophene groups provided another example of such advantages. (See, Examples 6 and 7, compare FIG. 6 and FIG. 1). The examples also show that modulating the extent of electron-donation on the π-conjugated electron donating group can influence the intensity of emission, a more quickly increasing rate of emission, and a shorter half-life for light emission (See, Examples 7 and 8). Specifically, reducing the electron-donating effect by directly substituting the r-conjugated electron donating group with an electron withdrawing group can result in reduced intensity, a slower emission rate increase, and a longer half-life for light emission (compare FIG. 7 and FIG. 6). Still, the effect of direct substitution with a thiophene, a type of π-conjugated electron donating group, provided significant advantages compared to the corresponding unsubstituted compound (compare FIG. 7 and FIG. 1). Further, the examples show that remote placement of a withdrawing or donating group (e.g., cyano or methoxy) does not outweigh the benefit of having a r-conjugated electron donating group placed directly onto the central, aryl ring that is bound to the dioxetane (See, Examples 9, 10, and 11; compare FIGS. 8, 9 and 10, to each other, and compare to FIG. 1). Thus, the π-conjugated electron donating group, e.g., a vinyl, aryl, or heteroaryl, can be further substituted and modified without destroying the improved luminescent properties.

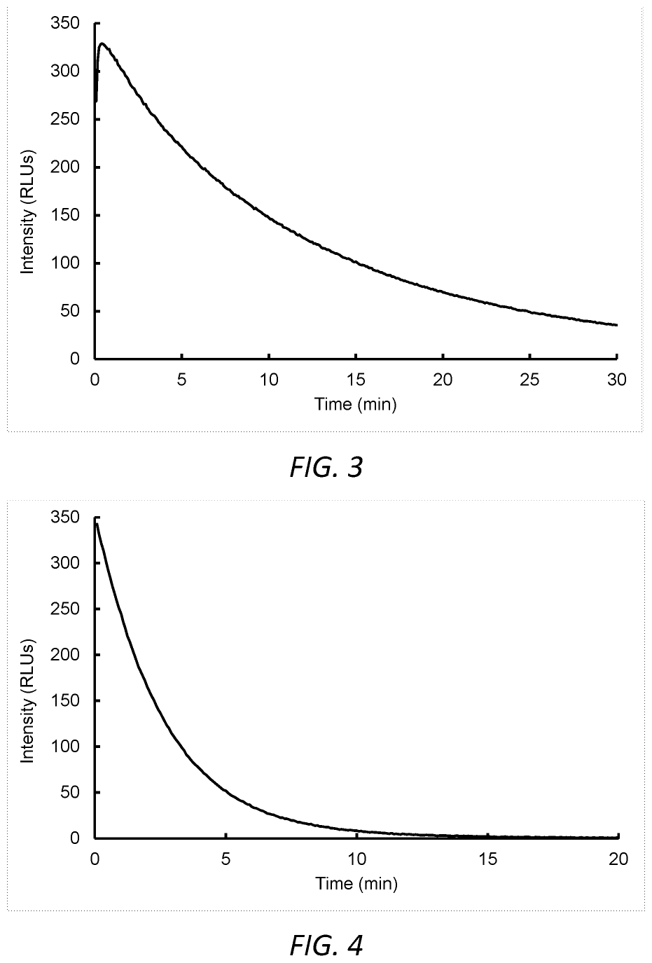

In comparison, compounds substituted with a group that was not r-conjugated, or not electron-donating resulted in a slower luminescence and a longer half-life for light emission (See Examples 2-4 and FIGS. 2-4). The effect in the case of chlorine was additive upon addition of a second chlorine. (compare FIGS. 2 and 3). Iodine, which is not a π-conjugated group and is neither strongly donating nor strongly withdrawing, resulted in a relatively small effect that did not significantly improve luminescence (See Example 4).

3-Phosphatephenyl derivatives were further prepared and tested under aqueous conditions by activation with alkaline-phosphatases (ALP) (See, Examples 12-16, and FIGS. 11-16). Two of the examples correspond to commercially available dioxetanes, namely Lumigen® PPD (Example 12) and Tropix CDP-Star® (Example 13), both lacking an electron-donating group on the central aromatic ring bound to the dioxetane. Both commercially available dioxetanes exhibited slow luminescence which did not reach a maximum light intensity or a steady-state intensity plateau for more than 15 minutes. In comparison, aqueous compositions of dioxetanes having a π-conjugated electron donating group demonstrated vastly improved speed and intensity of luminescence compared to the commercially-available dioxetanes (See, Examples 14-16, compare FIGS. 13-16 with FIG. 11 and FIG. 12).

Without intending to be limited to any theory, it surprisingly appears that increased electron density on the aromatic ring and π-conjugated substituents accelerate the rate limiting step of generating the transient excited-state species that undergoes chemilumescent decay. Based on these results, the speed and intensity of dioxetane chemiluminescence can be increased by use of substituents that are π-conjugated, electron-donating, or both. Moreover, an example was tested in aqueous conditions free of surfactant-based luminescence enhancers that provided surprisingly high-intensity luminescence. The compounds and compositions of the present disclosure thus represent a significant improvement over commercially-available dioxetanes.

### Example 18

Dioxetane compounds were prepared according to the following structures, consistent with the synthetic methods described herein:

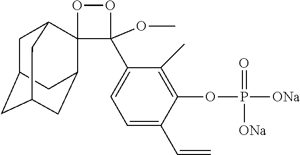

(D₂O ppm): 7.51-7.41 (m, 2H), 7.29-7.20 (m, 1H), 5.66 (d, J=17.2 Hz, 1H), 5.19 (d, J=11.2 Hz, 1H), 3.07 (s, 3H), 2.72 (s, 1H), 2.36 (s, 3H), 1.99 (s, 1H), 1.95-1.38 (m, 10H), 1.19-1.15 (m, 1H), 0.92-0.88 (m, 1H).

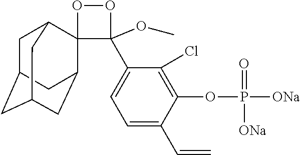

(D₂O) 7.56-7.49 (m, 2H), 7.26-7.19 (m, 1H), 5.71 (d, J=16.4 Hz, 1H), 5.26 (d, J=12.4 Hz, 1H), 3.10 (s, 3H), 2.66 (s, 1H), 2.01 (s, 1H), 1.67-1.43 (m, 10H), 1.39-1.18 (m, 1H), 1.10-1.06 (m, 1H).

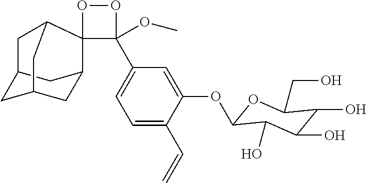 [SMILES: C=Cc1ccc(C2(OC)OOC23C2CC4CC(C2)CC3C4)cc1OC1OC(CO)C(O)C(O)C1O]

(CD₃OD) 7.53-7.50 (m, 1H), 7.40-6.85 (m, 3H), 5.74 (d, J=16.5 Hz, 1H), 5.29-5.24 (m, 1H), 4.96-4.91 (m, 1H), 4.20 (s, 1H), 4.12-4.02 (m, 1H), 3.85-3.75 (m, 5H), 3.17 (s, 3H), 2.98 (s, 1H), 2.10 (s, 1H), 1.85-1.44 (m, 10H), 1.24-1.19 (m, 1H), 1.0-0.85 (m. 1H).

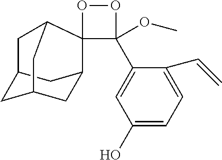 [SMILES: C=Cc1ccc(O)cc1C1(OC)OOC12C1CC3CC(C1)CC2C3]

(CDCl₃) 7.50-7.40 (m, 2H), 7.35-7.30 (s, 1H), 6.91 (s, 1H), 5.50 (d, J=16 Hz, 1H), 5.20 (d, J=16 Hz, 1H), 3.17 (s, 3H), 3.01 (s, 1H), 2.02 (s, 1H), 1.95-1.58 (m, 10H), 1.34-1.26 (m, 1H), 1.20-1.10 (m, 1H).

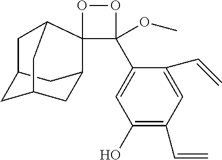 [SMILES: C=Cc1cc(C=C)c(C2(OC)OOC23C2CC4CC(C2)CC3C4)cc1O]

(CDCl₃) 7.80 (s, 1H), 7.46 (s, 1H), 7.35-7.25 (m, 1H), 7.10-6.90 (m, 1H), 5.87 (d, J=18 Hz, 1H), 5.54 (d, J=20 Hz, 1H), 5.41 (d, J=11.2 Hz, 1H), 5.21 (d, J=11.2 Hz, 1H), 3.18 (s, 3H), 3.01 (s, 1H), 2.15 (s, 1H), 2.05-1.46 (m, 10H), 1.26-1.24 (m, 1H), 1.20-1.16 (m, 1H).

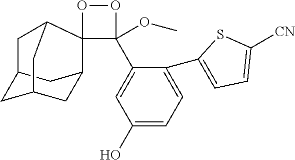 [SMILES: COC1(c2cc(O)ccc2-c2ccc(C#N)s2)OOC12C1CC3CC(C1)CC2C3]

(CDCl₃) 7.50 (d, J=4 Hz, 1H), 7.30-7.18 (m, 2H), 6.95-6.85 (m, 2H), 5.48 (br, 1H), 3.28 (s, 3H), 2.86 (s, 1H), 2.19 (s, 1H), 1.90-1.46 (m, 10H), 1.30-1.26 (m, 1H), 1.24-1.21 (m, 1H).

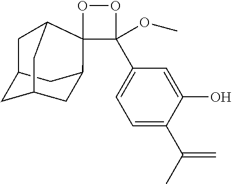 [SMILES: C=C(C)c1ccc(C2(OC)OOC23C2CC4CC(C2)CC3C4)cc1O]

(CDCl₃) 7.40-6.80 (m, 3H), 5.80 (s, 1H), 5.44 (s, 1H), 5.20 (s, 1H), 3.23 (s, 3H), 3.02 (s, 1H), 2.24 (s, 1H), 2.13 (s, 3H), 1.96-1.44 (m, 10H), 1.32-1.24 (m, 1H), 1.10-0.96 (m, 1H).

What is claimed is:

1. A compound of Formula I 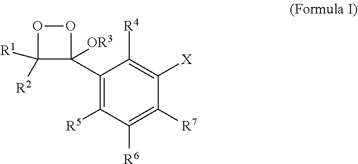
wherein
each of R¹ and R² is independently C₃-C₁₀ alkyl, or R¹ and R² taken together with the carbon to which they are attached provide a C₅-C₁₀ cycloalkyl ring;
R³ is C₁-C₁₀ alkyl, C₆-C₁₀ aryl, or heteroaryl;
each of R⁴, R⁵, R⁶ and R⁷ is independently H, Q, X, hydroxy, halogen, amino, thio, C₁-C₁₀ alkyl, C₂-C₁₀ alkenyl, C₂-C₁₀ alkynyl, C₁-C₁₀ alkyloxy, C₁-C₁₀ alkylamino, C₁-C₁₀ trialkylammonium salt, C₁-C₁₀ alkylthio, C₂-C₁₀ acyl, C₁-C₁₀ alkyloxycarbonyl, C₁-C₁₀ alkylaminocarbonyl, C₁-C₁₀alkylthiocarbonyl, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, C₁-C₁₀ alkylsulfinyl, C₁-C₁₀ alkylsulfonyl, arylthio, arylamino, arylsulfinyl, arylsulfonyl, arylcarbonyl, heteroarylcarbonyl, heteroaryloxy, heteroarylthio, heteroarylamino, heteroarylsulfinyl, heteroarylsulfonyl, cyano, nitro, trifluoromethyl, phosphonate, C₁-C₁₀ alkylphosphonate, C₁-C₁₀ alkylphosphinate, C₁-C₁₀ trialkylphosphonium salt, C₄-C₁₀ heterocycloamino, C₆-C₁₀ aryl, or π-excessive heteroaryl, and at least one of R⁴, R⁵, R⁶ and R⁷ is Q;
Q is a π-conjugated electron donating group;
X is —OH, —O-G, an —O⁻ salt, or a boron group having the structure 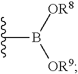
R⁸ and R⁹ is each independently H or C₁-C₁₀ alkyl, or R⁸ and R⁹ taken together with the boronate to which they are attached provide a C₂-C₁₀ cyclic boronate ester;
G is an alcohol protecting group.

2. The compound of claim 1, wherein R⁴, R⁵, R⁶ and R⁷ provide a net electron donating effect on the aromatic ring to which they are attached.

3. The compound of claim 1 having Formula I 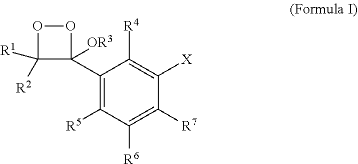
wherein
each of R¹ and R² is independently C₃-C₁₀ alkyl, or R¹ and R² taken together with the carbon to which they are attached provide a C₅-C₁₀ cycloalkyl ring;
R³ is C₁-C₁₀ alkyl, C₆-C₁₀ aryl, or heteroaryl;
each of R⁴, R⁵, R⁶ and R⁷ is independently H, Q, X, halogen, C₁-C₁₀ alkyl, hydroxy, C₁-C₁₀ alkyloxy, amino, C₁-C₁₀ alkylamino, thio, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, or heteroarylamino, and at least one of R⁴, R⁵, R⁶ and R⁷ is Q;
Q is an electron-donating group selected from C₂-C₁₀ alkenyl, C₂-C₁₀ heterocycloalkenyl, C₆-C₁₀ aryl, and heteroaryl;
X is —OH, —O-G, an —O⁻ salt, or a boron group having the structure 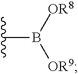
R⁸ and R⁹ is each independently H or C₁-C₁₀ alkyl, or R⁸ and R⁹ taken together with the boronate to which they are attached provide a C₂-C₁₀ cyclic boronate ester;
G is an alcohol protecting group.

4. The compound of claim 1, wherein Q is C₂-C₁₀ alkenyl, C₂-C₁₀ heterocycloalkenyl, C₆-C₁₀ aryl, or heteroaryl.

5. The compound of claim 4, wherein when Q is C₆-C₁₀ aryl it is substituted with one or more electron-donating groups.

6. The compound of claim 4, wherein when Q is C₂-C₁₀ alkenyl it is substituted with one or more electron-donating groups.

7. The compound of claim 4, wherein when Q is C₂-C₁₀ alkenyl it is free of electron-withdrawing groups.

8. The compound of claim 4, wherein when Q is C₂-C₁₀ alkenyl the vinylic positions and allylic positions, if present, are unsubstituted.

9. The compound of claim 4, wherein Q is free of electron-withdrawing groups.

10. The compound of claim 4, wherein Q is substituted with one or more electron-donating groups.

11. The compound of claim 4, wherein Q is unsubstituted vinyl.

12. The compound of claim 4, wherein Q is unsubstituted phenyl.

13. The compound of claim 4, wherein Q is phenyl substituted with one or more electron-donating groups.

14. The compound of claim 4, wherein Q is a π-excessive heteroaryl.

15. The compound of claim 14, wherein Q is thiophenyl, furanyl, pyrrolyl, benzothiophenyl, benzofuranyl, or indolyl.

16. The compound of claim 15, wherein Q is substituted or unsubstituted thiophen-2-yl or thiophen-3-yl.

17. The compound of claim 4, wherein R¹ and R² taken together with the carbon to which they are attached provide a spirocyclic bridged bicyclo or tricyclo group.

18. The compound of claim 17, wherein R¹ and R² taken together with the carbon to which they are attached provide a spirocyclic adamantane, norbornane, or bornane.

19. The compound of claim 1, wherein the aromatic ring to which X, Q, R⁴, R⁵, R⁶ and R⁷ attach is electron-enriched relative to an otherwise identical compound in which Q, R⁴, R⁵, R⁶ and R⁷ is H.

20. The compound of claim 1, wherein X is —OH.

21. The compound of claim 1, wherein X is —O-G, or a boron group having the structure

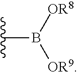

22. The compound of claim 1, wherein X is 4,4,5,5-tetramethyl-1,3,2-dioxaborolanyl or —B(OH)₂.

23. The compound of claim 1, wherein X is —O-G.

24. The compound of claim 1, wherein X is a group that generates an oxy anion upon chemical or enzymatic trigger.

25. The compound of claim 1, wherein G is an analyte-responsive protecting group.

26. The compound of claim 1, wherein G is trialkylsilyl, alkylarylsilyl, arylsulfonyl, dioxobenzyl, trityl, alkylcarbonate, phosphoryl, dihydropyranyl, tetrahydropyranyl, dihydrofuranyl, tetrahydrofuranyl, pyranosyl, pyranuronyl, furanosyl, acyl, benzoyl, or benzyl.

27. The compound of claim 26, wherein G is pyranosyl or pyranuronyl.

28. The compound of claim 27, wherein G is galactosyl, glucosyl, or glucuronyl.

29. The compound of claim 28, wherein G is β-galactosyl, β-glucosyl, or β-glucuronyl.

30. The compound of claim 29, wherein G is —PO₃H₂ or a salt or ester thereof.

31. The compound of claim 28, wherein G is 2,4-dinitrobenzenesulfonyl, 3,4,6-trimehyl-2,5-dioxobenzyl, 4-azidobenzyloxy, tert-butyldimethylsilyl, acetyl, pivaloyl, or a peptidase-cleavable moiety.

32. The compound of claim 1, wherein G comprises a divalent releasable linker and a terminal trialkylsilyl, alkylarylsilyl, arylbenzenesulfonyl, dioxobenzyl, trityl, alkylcarbonate, phosphoryl, dihydropyranyl, tetrahydropyranyl, dihydrofuranyl, tetrahydrofuranyl, pyranosyl, pyranuronyl, furanosyl, acyl, benzoyl, benzyl or boronate group.

33. The compound of claim 32, wherein the divalent releasable linker is 4-aminobenzyl, 4-(alkylamino)benzyl, 4-oxybenzyl, 4-(oxymethyl)benzyl, oxymethyl, aminomethyl, or alkylaminomethyl.

34. The compound of claim 1, wherein X has the structure:

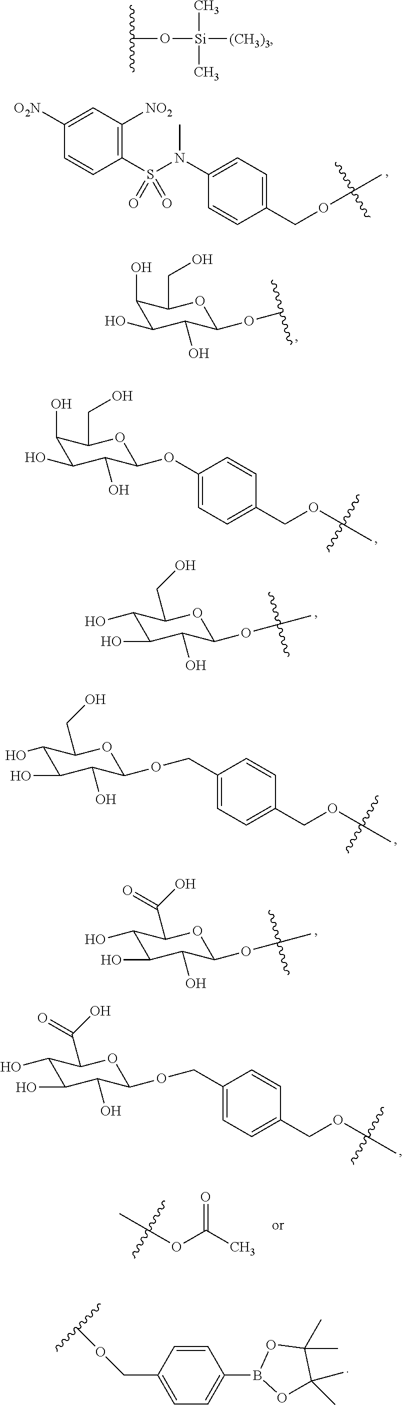

35. The compound of claim 1, wherein R³ is unsubstituted C₁-C₁₀ alkyl or C₁-C₁₀ alkyl substituted with one or more halogen, hydroxy, amino, thio, alkoxy, alkylamino, alkylthio, sulfate, or carboxylate.

36. The compound of claim 1, wherein each of R⁴, R⁵, R⁶ and R⁷ is independently H, Q, halogen, C₁-C₁₀ alkyl, hydroxy, C₁-C₁₀ alkyloxy, amino, C₁-C₁₀ alkylamino, thio, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, or heteroarylamino, and exactly one of R⁴, R⁵, R⁶ and R⁷ is Q.

37. The compound of claim 1, wherein one of R⁴, R⁵, R⁶ and R⁷ is Q and the remainder are H.

38. The compound of claim 1, wherein R⁷ is Q.

39. The compound of claim 37, wherein each of R⁴, R⁵, and R⁶ is H.

40. The compound of claim 1 having Formula II 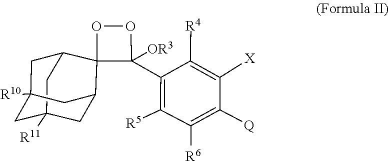
wherein each of R¹⁰ and R¹¹ is independently H or halogen.

41. The compound of claim 40 having Formula III 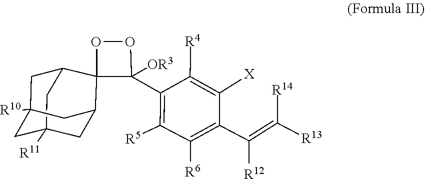
wherein
each of R¹² and R¹³ is independently H, halogen, C₁-C₁₀ alkyl, C₂-C₁₀ alkenyl, C₂-C₁₀ alkynyl, C₁-C₁₀ alkyloxy, C₁-C₁₀ alkylamino, C₁-C₁₀ trialkylammonium salt, C₁-C₁₀ alkylthio, C₂-C₁₀ acyl, C₁-C₁₀ alkyloxycarbonyl, C₁-C₁₀ alkylaminocarbonyl, C₁-C₁₀ alkylthiocarbonyl, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, C₁-C₁₀ alkylsulfinyl, C₁-C₁₀ alkylsulfonyl, arylthio, arylamino, arylsulfinyl, arylsulfonyl, arylcarbonyl, heteroarylcarbonyl, heteroaryloxy, heteroarylthio, heteroarylamino, heteroarylsulfinyl, heteroarylsulfonyl, cyano, nitro, trifluoromethyl, phosphonate, C₁-C₁₀ alkylphosphonate, C₁-C₁₀ alkylphosphinate, C₁-C₁₀ trialkylphosphonium salt, C₄-C₁₀ heterocycloamino, C₆-C₁₀ aryl, or π-excessive heteroaryl, or R¹² and R¹³ taken together with the carbons to which they are attached provide a C₅-C₁₀ cycloalkenyl, C₂-C₁₀ heterocycloalkenyl, C₆-C₁₀ aryl, or π-excessive heteroaryl;
R¹⁴ is H, halogen, C₁-C₁₀ alkyl, C₂-C₁₀ alkenyl, C₁-C₁₀ alkyloxy, C₁-C₁₀ alkylamino, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, or heteroarylamino, C₄-C₁₀ heterocycloamino, C₆-C₁₀ aryl, or π-excessive heteroaryl,
wherein R¹², R¹³ and R¹⁴ together have a net electron donating effect on the phenyl ring to which X is attached.

42. The compound of claim 41, wherein each of R¹² and R¹³ is independently H, C₁-C₁₀ alkyl, C₂-C₁₀ alkenyl, C₆-C₁₀ aryl, or π-excessive heteroaryl, or R¹² and R¹³ taken together with the carbons to which they are attached provide a C₅-C₁ cycloalkenyl, C₂-C₁₀ heterocycloalkenyl, C₆-C₁₀ aryl, or π-excessive heteroaryl; and
R¹⁴ is H, halogen, C₁-C₁₀ alkyl, C₂-C₁₀ alkenyl, C₁-C₁₀ alkyloxy, C₁-C₁₀ alkylamino, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, or heteroarylamino, C₄-C₁₀ heterocycloamino, C₆-C₁₀ aryl, or π-excessive heteroaryl.

43. The compound of claim 41, wherein each of R¹² and R¹³ is independently H, C₁-C₁₀ alkyl, C₂-C₁₀ alkenyl, C₆-C₁₀ aryl, or π-excessive heteroaryl, or R¹² and R¹³ taken together with the carbons to which they are attached provide a C₅-C₁ cycloalkenyl, C₂-C₁₀ heterocycloalkenyl, C₆-C₁₀ aryl, or π-excessive heteroaryl; and
R¹⁴ is H, C₁-C₁₀ alkyl, C₂-C₁₀ alkenyl, C₆-C₁₀ aryl, or π-excessive heteroaryl.

44. The compound of claim 41, wherein at least one of R¹², R¹³ and R¹⁴ is H.

45. The compound of claim 41, wherein at least two of R¹², R¹³ and R¹⁴ is H.

46. The compound of claim 41 having a structure according to one or more of the following formulae: 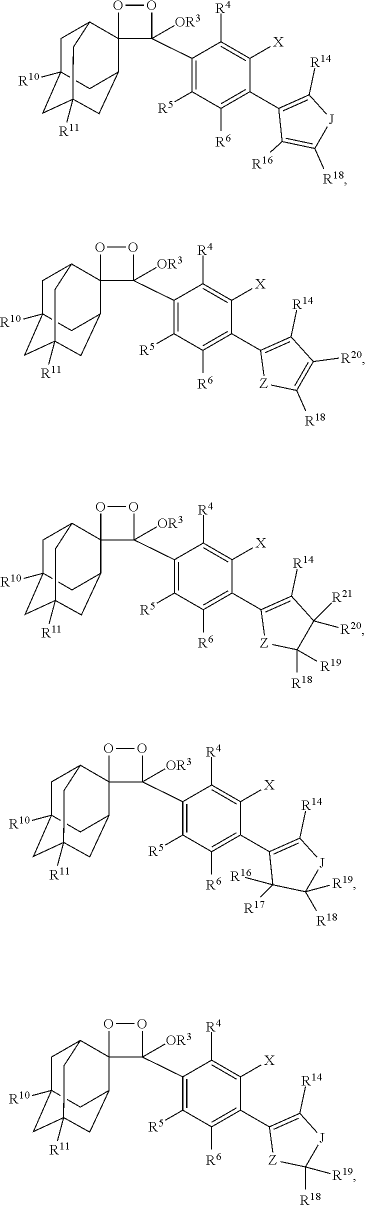 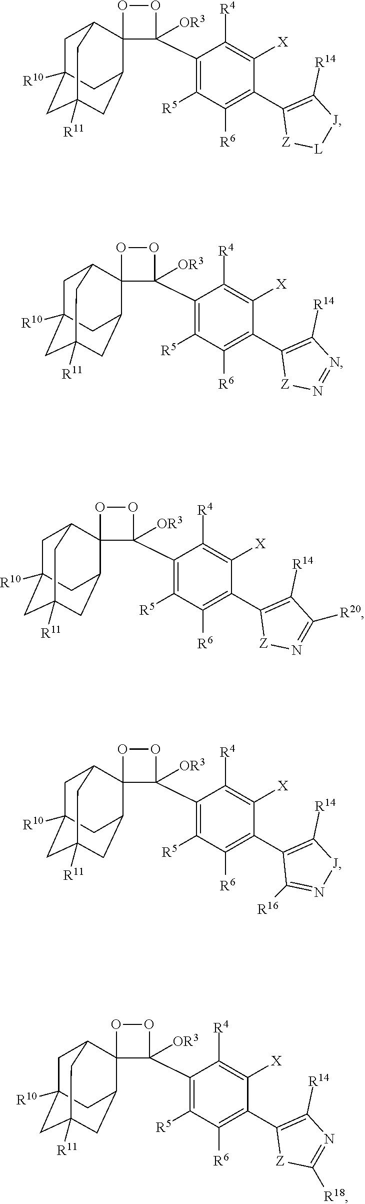 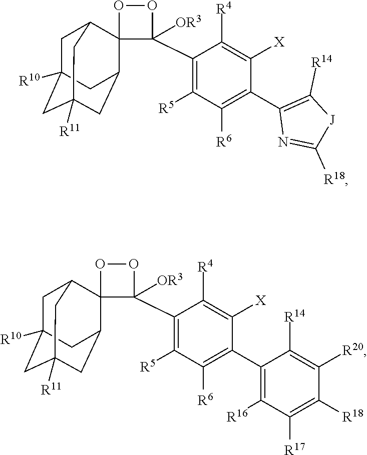 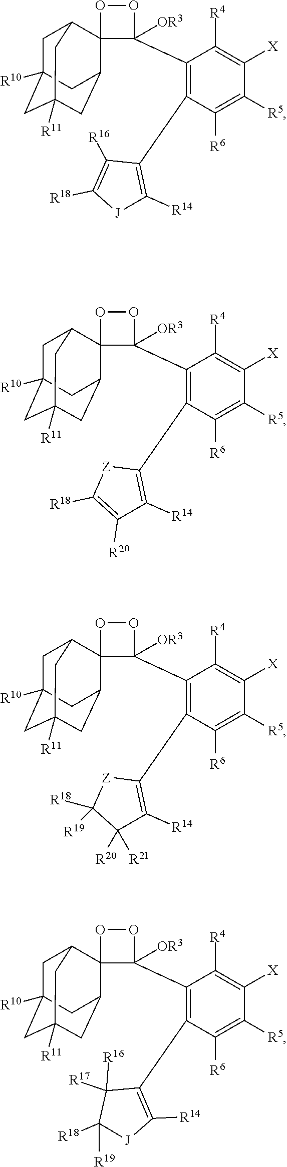 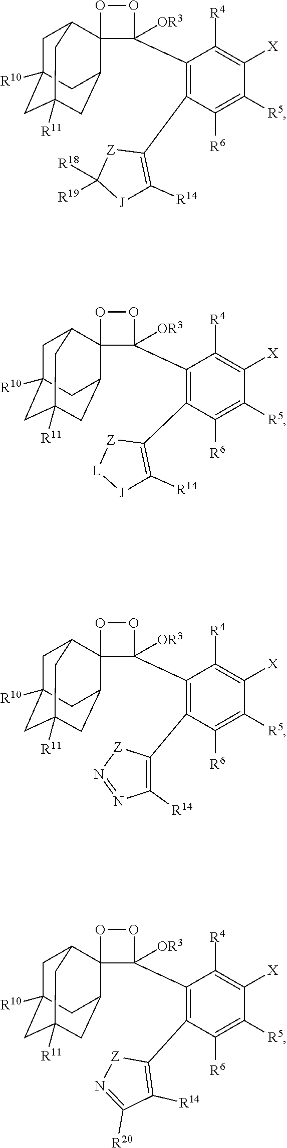 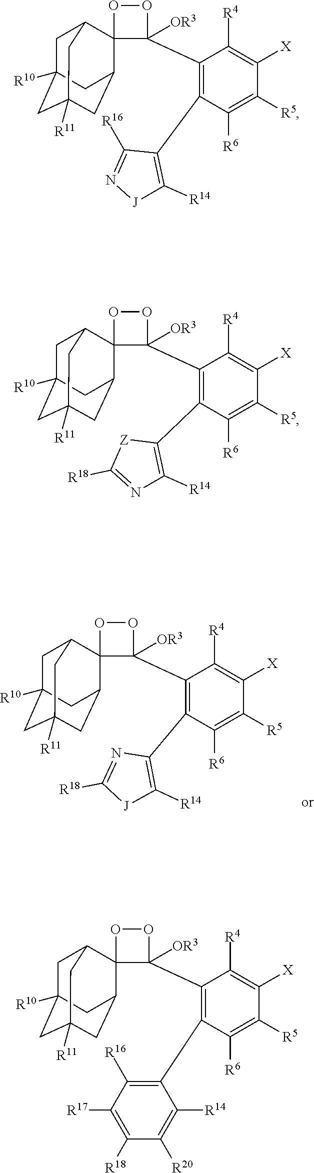
wherein
each of Z, L and J is S, O, Se, NR¹⁵, or (CR¹⁶R¹⁷)ₙ;
each R¹⁵ is independently H, alkyl, acyl, benzyl, alkyloxycarbonyl, arylsulfonyl; and
each of R¹⁴, R¹⁶, R¹⁷, R¹⁸, R¹⁹, R²⁰ and R²¹, if present, is independently H, halogen, C₁-C₁₀ alkyl, C₂-C₁₀ alkenyl, C₁-C₁₀ alkyloxy, C₁-C₁₀ alkylamino, C₁-C₁₀ trialkylammonium salt, C₁-C₁₀ alkylthio, C₂-C₁₀ acyl, C₁-C₁₀ alkyloxycarbonyl, C₁-C₁₀ alkylaminocarbonyl, C₁-C₁₀ alkylthiocarbonyl, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, C₁-C₁₀ alkylsulfinyl, C₁-C₁₀ alkylsulfonyl, arylthio, arylamino, arylsulfinyl, arylsulfonyl, arylcarbonyl, heteroarylcarbonyl, heteroaryloxy, heteroarylthio, heteroarylamino, heteroarylsulfinyl, heteroarylsulfonyl, cyano, nitro, trifluoromethyl, phosphonate, C₁-C₁₀ alkylphosphonate, C₁-C₁₀ alkylphosphinate, C₁-C₁₀ trialkylphosphonium salt, C₄-C₁₀ heterocycloamino, C₆-C₁₀ aryl, or π-excessive heteroaryl, or any two of R¹⁴, R¹⁶, R¹⁷, R¹⁸ and R²⁰ taken together with the carbons to which they are attached provide a C₅-C₁₀ cycloalkenyl, C₂-C₁₀ heterocycloalkenyl, C₆-C₁₀ aryl, or π-excessive heteroaryl.

47. The compound of claim 46, wherein each of R¹⁴, R¹⁶, R¹⁷, R¹⁸, R¹⁹, R²⁰ and R²¹, if present, is independently H, halogen, C₁-C₁₀ alkyl, C₂-C₁₀ alkenyl, C₁-C₁₀ alkyloxy, C₁-C₁₀ alkylamino, C₁-C₁₀ alkylthio, C₂-C₁₀ acyloxy, C₂-C₁₀ acylamino, C₂-C₁₀ acylthio, C₁-C₁₀ alkylcarbonate, C₁-C₁₀ alkylcarbamate, C₁-C₁₀ carbamido, aryloxy, arylthio, arylamino, heteroaryloxy, heteroarylthio, or heteroarylamino, C₄-C₁₀ heterocycloamino, C₆-C₁₀ aryl, or π-excessive heteroaryl, or any two of R¹⁴, R¹⁶, R¹⁷, R¹⁸ and R²⁰ taken together with the carbons to which they are attached provide a C₅-C₁₀ cycloalkenyl, C₂-C₁₀ heterocycloalkenyl, C₆-C₁₀ aryl, or π-excessive heteroaryl.

48. The compound of claim 46, wherein at least one of R¹⁴, R¹⁶, R¹⁷, R¹⁸ and R²⁰ is an electron donating group.

49. The compound of claim 46, wherein each of R¹⁴, R¹⁶, R¹⁸ and R²⁰, if present, is hydrogen.

50. The compound of claim 1 having Formula IV-XV.

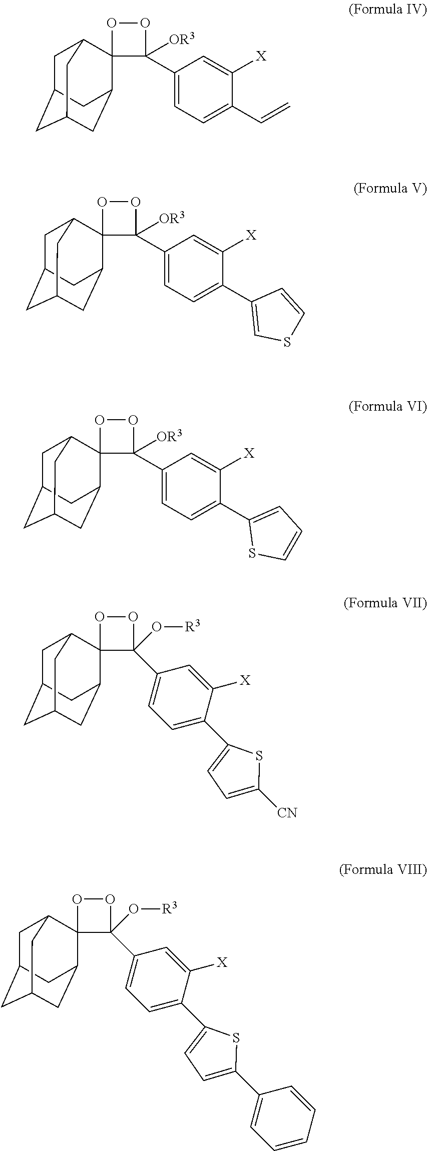

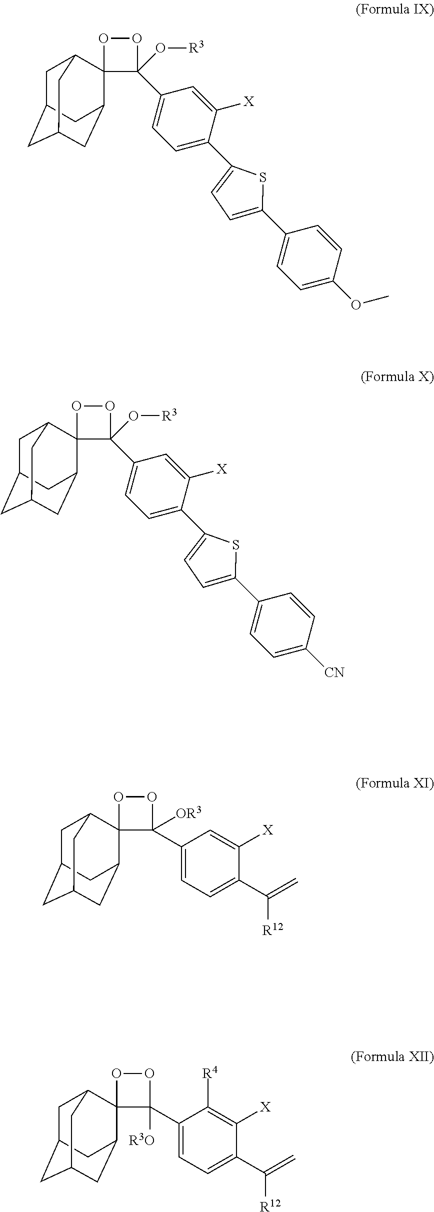

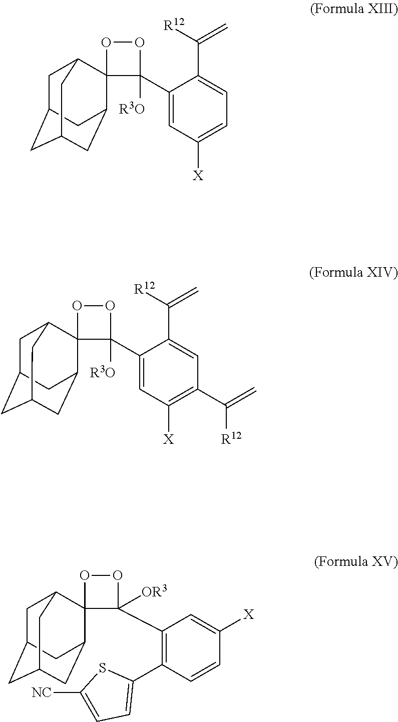

51. The compound of claim 1 having the structure: 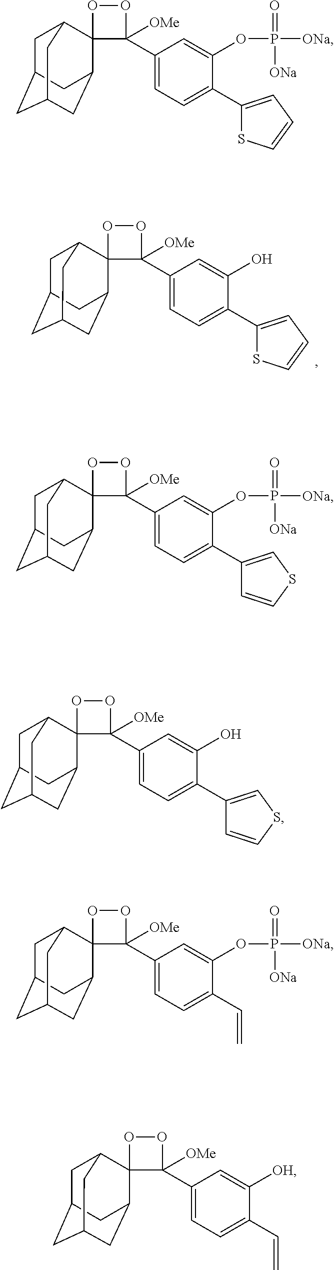 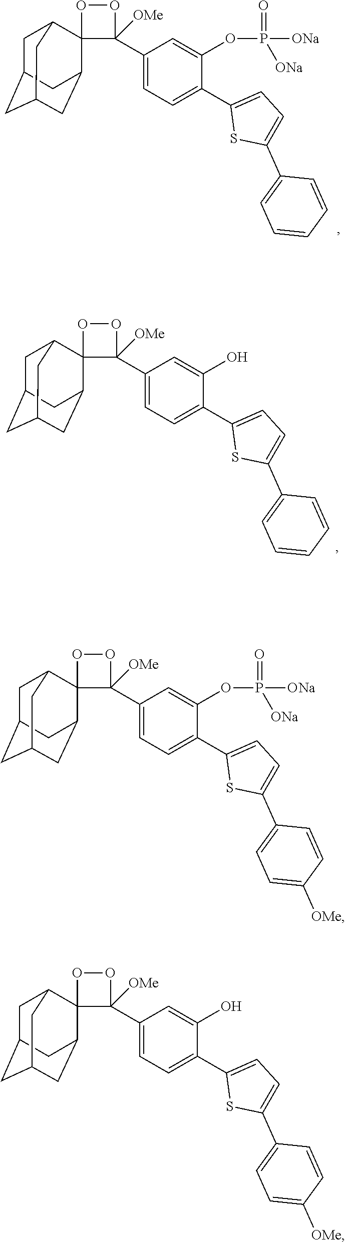 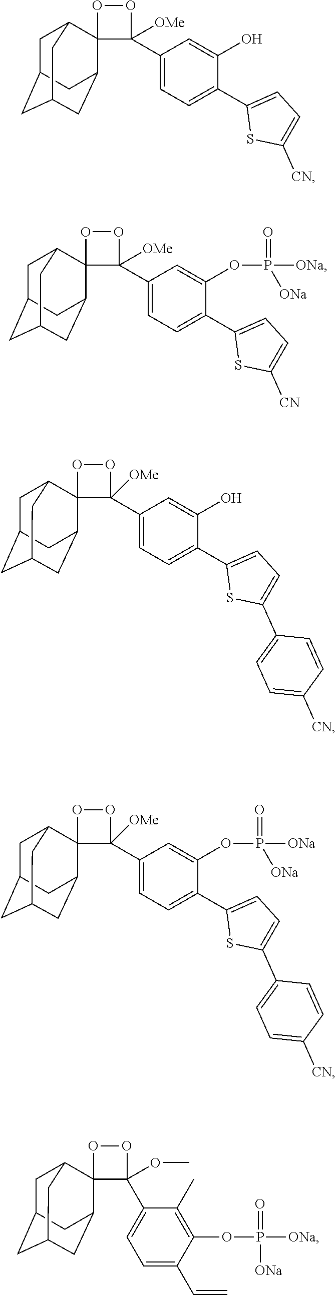 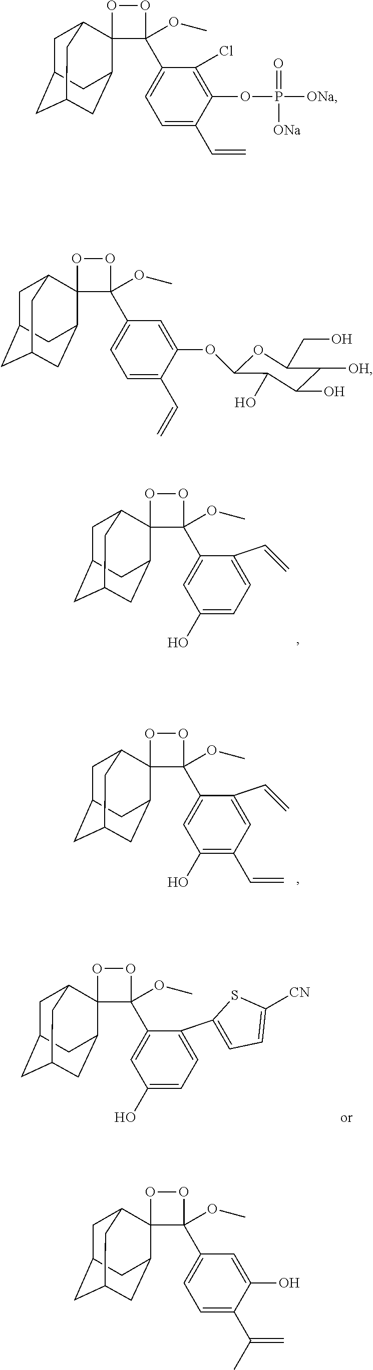
or a salt thereof.

52. A compound having the structure: 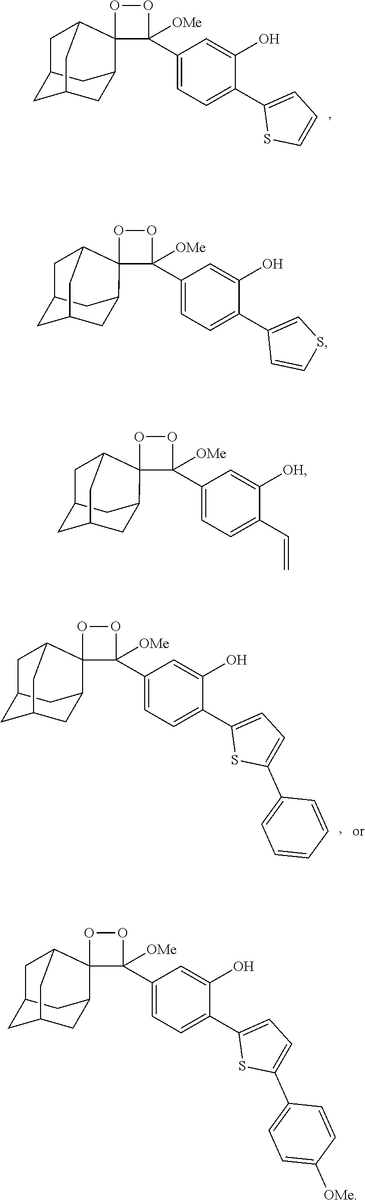 [SMILES: COC1(c2ccc(-c3cccs3)c(O)c2)OOC12C1CC3CC(C1)CC2C3 ; COC1(c2ccc(-c3ccsc3)c(O)c2)OOC12C1CC3CC(C1)CC2C3 ; C=Cc1ccc(C2(OC)OOC23C2CC4CC(C2)CC3C4)cc1O ; COC1(c2ccc(-c3ccc(-c4ccccc4)s3)c(O)c2)OOC12C1CC3CC(C1)CC2C3 ; COc1ccc(-c2ccc(-c3ccc(C4(OC)OOC45C4CC6CC(C4)CC5C6)cc3O)s2)cc1]
or a salt thereof.

53. A compound having the structure:

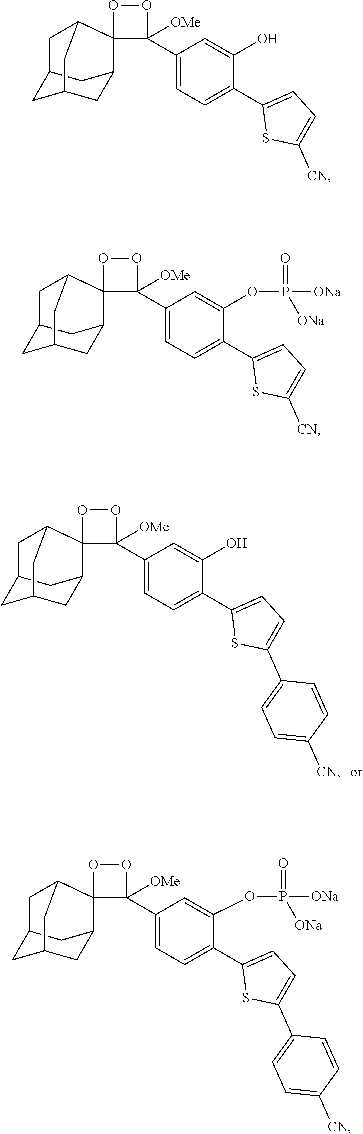

54. The compound of claim 1 having Formula Ila or IIb 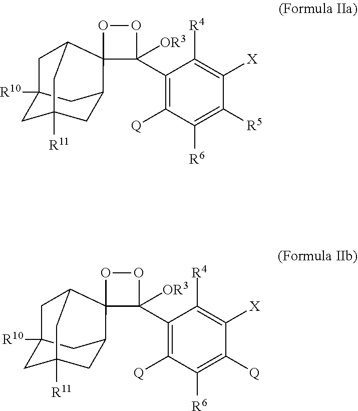
wherein each of R¹⁰ and R¹¹ is independently H or halogen.

55. The compound of claim 1 having Formula IIIa or IlIb 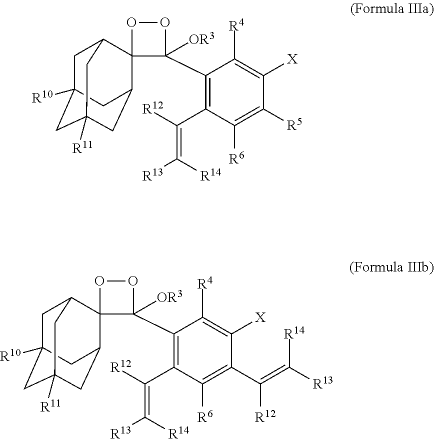
wherein each of R¹⁰ and R¹¹ is independently H or halogen.

56. A composition comprising a compound of any one of claims 1-55.

57. The composition of claim 56, wherein the composition is substantially free of surfactant-based luminescence enhancers.

58. The composition of claim 56, wherein the composition is substantially free of surfactants.

59. The composition of claim 56, wherein the composition is substantially free of surfactants having an acyclic alkyl group or an aromatic group and one or more quaternary ammonium salt, pyridinium salt, quaternary phosphonium surfactant salt, ethyleneglycol.

60. The composition of claim 56, wherein the composition is substantially free of cetrimonium bromide (CTAB), cetylpyridinium chloride (CPC), benzalkonium chloride (BAC), benzethonium chloride (BZT), dimethyldioctadecylammonium chloride, dioctadecyldimethylammonium bromide (DODAB), α′-tributylphosphonium-p-xylene dichloride, poly(vinylbenzyltributylphosphonium chloride) (TBE), and poly(vinylbenzyltrioctylphosphonium chloride).

61. The composition of claim 56, further comprising an amine buffer.

62. The composition of claim 56 in the form of an aqueous composition.

63. The composition of claim 56 having a peak luminescent intensity of greater than 1000 photons/sec and a T_{1/2} of 30 seconds or less at 37° C. upon treatment with pH 9.7 buffer, and wherein the composition is substantially free of surfactants.

64. An aqueous chemiluminescent composition comprising one or more chemiluminescent dioxetane compound and having a peak luminescent intensity of greater than 1000 photons/sec and a T_{1/2} of 30 seconds or less at 37° C. upon treatment with pH 9.7 buffer, and wherein the composition is substantially free of surfactant-based luminescence enhancers.

65. A method of detecting an analyte in a sample, comprising contacting the sample with a compound of any one of claims 1-55 and monitoring the sample for luminescence.

66. The method of claim 65, further comprising measuring the intensity of a resulting luminescence and correlating the intensity to the presence of the analyte.

67. The method of claim 65, wherein the contacted sample is monitored for less than 1 minute.

68. The method of claim 65, wherein the analyte is alkaline phosphatase.

69. The method of claim 65, wherein the analyte is an oxidizing agent.

70. The method of claim 69, wherein the analyte is hydrogen peroxide.

71. The method of claim 69, wherein the analyte is glutathione.

72. The method of claim 69, wherein the analyte is fluoride.

73. The method of claim 69, wherein the analyte is a base.

74. The method of claim 69, wherein the sample is an aqueous sample having a pH of about 4 to about 12.

75. The method of claim 74, wherein the sample is an aqueous sample having a pH of about 8 to about 12.

76. A kit for determining the presence of an analyte, the kit comprising the compound of any one of claims 1-55 or the composition of any one of claims 56-64.
